(19) United States
(12) Patent Application Publication
(10) Pub. No.: US 2009/0203035 A1
(43) Pub. Date: Aug. 13, 2009

(54) FLUORESCENT PROTEINS WITH INCREASED PHOTOSTABILITY

(75) Inventors: Nathan C. Shaner, Seaside, CA (US); Michael Z. Lin, San Diego, CA (US); Michael McKeown, La Jolla, CA (US); Roger Y. Tsien, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/259,089

(22) Filed: Oct. 27, 2008

Related U.S. Application Data
(60) Provisional application No. 60/982,985, filed on Oct. 26, 2007.

(51) Int. Cl. G01N 33/53; C07K 14/00; C07H 21/00; C12N 15/74; C12N 5/06; C12Q 1/02

(52) U.S. Cl. 435/7.1; 530/350; 530/402; 536/23.1; 435/320.1; 435/325; 435/29

(57) ABSTRACT

The present invention relates to novel fluorescent protein variants of DsRed and eqFP578. Fluorescent protein variants having increased photostability and/or having reversible photoswitching behavior, as well as polynucleotides encoding such variants are provided herein. Methods of using these novel fluorescent protein variants and methods for constructing other fluorescent protein variants having increased photostability are also provided by the present invention.

### CROSS-REFERENCES TO RELATED APPLICATIONS

The present application claims priority to U.S. Ser. No. 60/982,985, filed Oct. 26, 2007, herein incorporated by reference in its entirety.

### STATEMENT AS TO RIGHTS TO INVENTIONS MADE UNDER FEDERALLY SPONSORED RESEARCH OR DEVELOPMENT

The present invention was made under NIH Grant Nos. GM072033 and NS027177. The Government has certain rights in this invention.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The identification and isolation of fluorescent proteins in various organisms, including marine organisms, has provided a valuable tool to molecular biology. The green fluorescent protein (GFP) of the jellyfish Aequorea victoria, for example, has become a commonly used reporter molecule for examining various cellular processes, including the regulation of gene expression, the localization and interactions of cellular proteins, the pH of intracellular compartments, and the activities of enzymes.

The usefulness of Aequorea GFP has led to the identification of numerous other fluorescent proteins in an effort to obtain proteins having different useful fluorescence characteristics. In addition, spectral variants of Aequorea GFP have been engineered, thus providing proteins that are excited or fluoresce at different wavelengths, for different periods of time, and under different conditions. The identification and cloning of a red fluorescent protein from Discosoma coral, termed DsRed or drFP583, has raised a great deal of interest due to its ability to fluoresce at red wavelengths.

The DsRed from Discosoma (Matz et al., Nature Biotechnology, 17:969-973 (1999)) holds great promise for biotechnology and cell biology as a spectrally distinct companion or substitute for the green fluorescent protein (GFP) from the Aequorea jellyfish (Tsien, Ann. Rev. Biochem., 67:509-544 (1998)). GFP and its blue, cyan, and yellow variants have found widespread use as genetically encoded indicators for tracking gene expression and protein localization and as donor/acceptor pairs for fluorescence resonance energy transfer (FRET). Extending the spectrum of available colors to red wavelengths would provide a distinct new label for multicolor tracking of fusion proteins and together with GFP (or a suitable variant) would provide a new FRET donor/acceptor pair that should be superior to the currently preferred cyan/yellow pair (Mizuno et al., Biochemistry, 40:2502-2510 (2001)).

In the past several years, substantial progress has been made in the development of monomeric or dimeric fluorescent proteins covering the entire visual spectrum (Campbell, R. E. et al., Proc Natl Acad Sci USA, 99:7877-7882 (2002); Shaner, N. C. et al., Nat Biotechnol, 22:1567-1572 (2004); Chudakov, D. M. et al., Nat Biotechnol, 22:1435-1439 (2004); Griesbeck, O. et al., J Biol Chem, 276:29188-29194 (2001); Habuchi, S. et al., Proc Natl Acad Sci USA, 102:9511-9516 (2005); Karasawa, S. et al., Biochem J, 381:307-312 (2004); Nagai, T. et al., Nat Biotechnol, 20:87-90 (2002); Nguyen, A. W. and Daugherty, P. S., Nat Biotechnol, 23:355-360 (2005); Rizzo, M. A. et al., Nat Biotechnol, 22:445-449 (2004); Wiedenmann, J. et al., Proc Natl Acad Sci USA, 101:15905-15910 (2004); Zapata-Hommer, O. and Griesbeck, O., BMC Biotechnol, 3, 5 (2003); Ai, H. W. et al., Biochem J. 400:531-540 (2006); Merzlyak, E. M. et al., Nat Methods, 4:555-557 (2007)), but while brightness and wavelength have been a primary concern, photostability has generally been an afterthought (with the notable exception of mTFP1 (Ai, H. W. et al., Biochem J, 400:531-540 (2006)). As a result, many novel fluorescent protein variants have relatively poor photostability.

All organic fluorophores undergo irreversible photobleaching during prolonged illumination. While fluorescent proteins typically bleach at a substantially slower rate than many small molecule dyes, lack of photostability remains an important limiting factor for experiments requiring large numbers of images of single cells. Screening methods focusing solely on brightness or wavelength are highly effective in optimizing both properties, but the absence of selective pressure for photostability in such screens leads to unpredictable photobleaching behavior in the resulting fluorescent proteins. The first-generation monomeric red fluorescent protein, mRFP1 (U.S. Pat. No. 7,005,511), while reasonably bright, suffered a substantial decrease in photostability compared to its ancestor, Discosoma sp. red fluorescent protein (DsRed). In subsequent generations of mRFP1 variants (the “mFruits”) (U.S. Pat. No. 7,157,566), serendipitous enhancement in photostability was observed in some variants, suggesting that it would be possible to apply directed evolution strategies to this property as well.

To extend the utility of the existing set of fluorescent proteins, having optimized them for many other properties, the present invention advantageously provides novel screening methods that additionally queries photostability in a medium-Throughput format. This selection scheme allow for simultaneous selection of the most photostable mutants that also maintain an acceptable level of fluorescence emission at the desired wavelength, minimizing the tradeoff of desirable properties that frequently results from single-parameter screens. The present invention also fulfills a need in the art by providing photostable fluorescent protein variants.

### BRIEF SUMMARY OF THE INVENTION

In a first embodiment, the present invention provides novel fluorescent protein variants with increased photostability. In one embodiment, the novel fluorescent protein variants of the present invention have an increased photobleaching half-life. In certain embodiments, these fluorescent proteins are mutants of wild type fluorescent proteins. In some embodiments of the invention, the novel fluorescent proteins are generated through directed evolution of one or more photostability properties.

In certain embodiments, the novel photostable fluorescent protein variants provided by the present invention are mutants of a fluorescent protein selected from AvGFP, DsRed, eqFP578, eqFP611, and the like. In other embodiments, the fluorescent proteins having increased photostability are mutants of spectral variants, such as mOrange, mCherry, TagRFP, and the like. In a particular embodiment of the invention, the photostable fluorescent protein variants include mApple0.1, mApple0.5, mApple, mOrange2, TagRFP-T, and mutants thereof.

In a second embodiment, the present invention provides nucleic acids encoding fluorescent proteins with increased photostability. In certain embodiments, the encoded fluorescent proteins have an increased photobleaching half-life. In other embodiments, the invention provides nucleic acids encoding photostable fluorescent protein variants that are mutants of wild type fluorescent proteins. In still other embodiments, the novel fluorescent proteins provided by the invention are generated through directed evolution of one or more photostability properties. Nucleic acids provided by the present invention may include constructs for in vivo or in vitro protein expression, including prokaryotic and eukaryotic expression vectors. Nucleic acid sequences encoding the fluorescent protein variants of the invention may further comprise regulatory sequences, such as transcriptional regulators, which may be functionally linked to the coding sequences. In yet other embodiments, the present invention provides nucleic acids and vectors encoding fusion fluorescent proteins having increased photostability or tandem fluorescent proteins having increased photo stability.

In a third embodiment, the present invention provides host cells comprising a nucleic acid or vector encoding for a fluorescent protein variant having increased photostability. Suitable host cells include eukaryotic cells, such as mammalian cells or yeast cells, and prokaryotic cells, such as bacterial cells. In yet other embodiments, the present invention provides host viral particles comprising a nucleic acid or vector encoding for a photostable fluorescent protein variant. Suitable viral particles may comprise ssRNA, dsRNA, ssDNA, or dsDNA viruses. The host cells or virus particles of the invention may also comprise fusion or tandem fluorescent protein variants having increased photostability.

In a fourth embodiment, the present invention provides methods of detecting the expression of a protein, detecting the localization of a protein, detecting the motility of a protein, or detecting a protein-protein interaction using a nucleic acid or vector encoding a fluorescent protein variant having a increased photostability. The methods provided herein may alternatively comprise the use of nucleic acids or vectors encoding for a fusion or tandem fluorescent protein variant having increased photostability. In certain embodiments of the invention, these methods comprise the use of a nucleic acid encoding for a mutant of an AvGFP, DsRed, EqRFP, or similar wild type fluorescent protein, which has increased photostability with respect to the wild type protein. In a particular embodiment, the methods of the invention comprise the use of a nucleic acid encoding for a variant fluorescent protein selected from mApple0.1, mApple0.5, mApple, mOrange2, TagRFP-T, and mutants thereof.

In a fifth embodiment, the present invention provides methods of detecting the expression of a protein, detecting the localization of a protein, detecting the motility of a protein, or detecting a protein-protein interaction using a fluorescent protein variant having a increased photostability. The methods provided herein may alternatively comprise the use of a fusion or tandem fluorescent protein variant having increased photostability. In certain embodiments of the invention, these methods comprise the use of a mutant of an AvGFP, DsRed, EqFP578, eqFP611, or similar wild type fluorescent protein, which has increased photostability with respect to the wild type protein. In a particular embodiment, the methods of the invention comprise the use of a variant fluorescent protein selected from mApple0.1, mApple0.5, mApple, mOrange2, TagRFP-T, and mutants thereof.

In a sixth embodiment, the invention provides fluorescent protein variants having increased photostability, which are useful for fluorescent resonance energy transfer (FRET). In some embodiments, the fluorescent protein variants of the invention may demonstrate FRET with other protein variants provided by the present invention. In other embodiments, the fluorescent protein variants of the invention may demonstrate FRET with fluorescent proteins not having increased photostability. In yet other embodiments of the invention, the photostable fluorescent protein variants that are useful for FRET may comprise fusion or tandem fluorescent proteins. Tandem fluorescent proteins of the invention may be capable of intermolecular FRET, intramolecular FRET, or both.

In a seventh embodiment of the invention, methods of developing fluorescent protein variants having increased photostability are provided. In certain embodiments, these methods comprise screening mutant fluorescent proteins for increased photostability. In other embodiments, these methods comprise the steps of performing protein evolution on a parent fluorescent protein and selecting a variant fluorescent protein having an increased photostability with respect to the parent fluorescent protein. In a particular embodiment, the step of performing protein evolution comprises alternating or intermittent mutagenesis and selection for photostability. Mutagenesis of nucleic acids encoding fluorescent proteins may comprise random mutagenesis, directed mutagenesis, or both. In another particular embodiment, the methods of selecting for increased photostability, comprise selecting for increased resistance to photobleaching. In certain embodiments of the invention, selection for increased photostability may be performed using a solar simulator.

In an eighth embodiment, the present invention provides fluorescent protein variants demonstrating reversible photoswitching behavior and methods of generating such variants. In one embodiment, the photoswitching fluorescent protein variants of the invention are mutant proteins of an AvGFP, DsRed, eqFP578, eqFP611, or similar fluorescent protein. In a particular embodiment, the present invention provides a photoswitching fluorescent protein comprising an amino acid sequence of mApple0.1 (SEQ ID NO:8), mApple0.5 (SEQ ID NO:9), or mApple (SEQ ID NO:10). In one embodiment, the present invention provides fluorescent protein variants with reversible photoswitching behavior that are useful for nanoscale spatial resolution (“nanoscopy”). As such, the invention also provides fusion proteins and tandem fluorescent protein variants with reversible photoswitching behavior that are useful as probes in nanoscopy. In yet another specific embodiment of the invention, fluorescent protein variants, which are particularly useful for photoactivated localization microscopy with independently running acquisition (PALMIRA), are provided.

### BRIEF DESCRIPTION OF THE DRAWINGS

FIG. 1. Characterization of excitation and emission spectra for mApple, mOrange2, and TagRFP-T. Excitation (measured at emission maximum, solid lines) and emission (measured at excitation maximum, dotted lines) spectra for (a) mApple and (b) mOrange2, and (c) excitation (measured at emission maximum, dotted line) and emission (measured at excitation maximum, purple solid line; measured with 480 nm excitation, green dashed line) spectra for TagRFP-T; (d) absorbance spectra for mApple (red dotted line), mOrange2 (orange dashed line) and TagRFP-T (purple solid line).

FIG. 2. Analysis of photostability mutations. Normalized arc lamp photobleaching curves for (a) mRFP1 (solid line) and its Q64H+F99Y mutant (dotted line), (b) mOrange under normal (solid line) and O₂-free (dotted line) conditions, (c) mOrange2 under normal (solid line) and O₂-free (dotted line) conditions, (d) TagRFP (blue lines) and TagRFP-T (red lines) under normal (solid lines) and O₂-free (dotted line) conditions, and (e) mOrange and mOrange2 under normoxic and O₂-free conditions.

FIG. 3. Fluorescence imaging of mOrange2 subcellular targeting fusions. Widefield fluorescence images of mOrange2 chimeras in N- and C-Terminal fusions. N-Terminal fusion constructs (linker amino acid length indicated after fusion protein name): (A) mOrange2-Keratin-17 (human cytokeratin 18); (B) mOrange2-Cx26-7 (rat β-2 connexin-26); (C) mOrange2-Golgi-7 (N-Terminal 81 amino acids of human β-1,4-galactosyltransferase); (D) mOrange2-vimentin-7 (human); (E) mOrange2-EB3-7 (human microtubule-associated protein; RP/EB family); (F) mOrange2-mitochondria-7 (human cytochrome C oxidase subunit VIII); (G) mOrange2-paxillin-22 (chicken); (H) mOrange2-α-actinin-19 (human non-muscle); C-Terminal fusion constructs: (I) mOrange2-Lamin B1-10 (human); (J) mOrange2-β-Actin-7 (human); (K) mOrange2-lysosomes-20 (rat lysosomal membrane glycoprotein 1); (L) mOrange2-peroxisomes-2 (peroximal targeting signal 1); (M) mOrange2 β-Tubulin-6 (human); (N) mOrange2-Fibrillarin-7 (human); (O) mOrange2-vinculin-23 (human); (P) mOrange2-Clathrin Light Chain-15 (human). (Q-U) Laser scanning confocal images of HeLa cells expressing mOrange2-H2B-6 (N-Terminal fusion; human) progressing through (Q) interphase; (R) prophase; (S) prometaphase; (T) metaphase; (U) early anaphase. The cell line used for expressing mOrange2 fusion vectors was Gray fox lung fibroblast cells (FoLu) in panels (E) and (J), and human cervical adenocarcinoma cells (HeLa) in the remaining panels.

FIG. 4. Comparison of mOrange2, mKO, and tdTomato fusions in microtubules and gap junctions. (A-C) Widefield fluorescence images of HeLa cells expressing an identical human α-Tubulin (C-Terminus; 6-amino acid linker) localization construct fused to: (A) mOrange2; (B) mKO; (C) tdTomato. 100× magnification; Bar=10 μm. (D-F) HeLa cells expressing an identical rat α-1 connexin-43 (N-Terminus; 7-amino acid linker) localization construct fused to (D) mOrange2; (E) mKO; (F) tdTomato. 60× magnification; Bar=10 μm.

FIG. 5. The amino acid sequence (SEQ ID NO:1) and coding nucleic acid sequence (SEQ ID NO:2) for TagRFP.

FIG. 6. The amino acid sequence of mOrange (SEQ ID NO:3).

FIG. 7. A nucleic acid sequence encoding the mOrange protein (SEQ ID NO:4).

FIG. 8. Reversible photoswitching in mApple0.5. 10 cycles of continuous arc lamp illumination with 10% neutral density filter for four seconds (solid lines, individual data points shown), with 30 seconds of darkness between cycles (dotted lines) (normalized intensity versus actual exposure time). All data points are normalized to the initial image intensity (at time 0); the progressive slight decreases in recovered intensity after each cycle are presumably due to small amounts of irreversible photobleaching or fatigue. mApple0.5 is the immediate precursor to mApple which lacks the external mutations R17H, K92R, S147E, T175A, and T202V.

FIG. 9. Laser scanning confocal microscopy photobleaching curves. Comparison of photobleaching curves. (a) Arc-lamp photobleaching curves for mRFP1, EGFP, mCherry, tdTomato, mOrange, mKO, TagRFP, mApple, mOrange2 and TagRFP-T, as measured for purified protein and plotted as intensity versus normalized total exposure time with an initial emission rate of 1,000 photons/s per molecule. (b) Normalized laser scanning confocal microscopy bleaching curves for the same proteins (except for EGFP, which in this case is the monomeric A206K variant) fused to histone H2B and imaged in live cells. The time axis represents normalized total imaging time for an initial scan-averaged emission rate of 1,000 photons/s per molecule.

FIG. 10. Fluorescence imaging of TagRFP-T subcellular targeting fusions. (A-G) N-Terminal fusion constructs (linker amino acid length indicated by the numbers): TagRFP-T-N1 (A; N-Terminal fusion cloning vector; expression in nucleus and cytoplasm with no specific localization); TagRFP-T-7-cytochrome c oxidase (B; mitochondria human cytochrome c oxidase subunit VIII); TagRFP-T-6-histone H2B (C; human; showing two interphase nuclei and one nucleus in early anaphase); TagRFP-T-7-β-1,4-galactosyltransferase (D; golgi; N-Terminal 81 amino acids of human β-1,4-galactosyltransferase); TagRFP-T-7-vimentin (E; human); TagRFP-T-7-Cx43 (F; rat α-1 connexin-43); and TagRFP-T-7-zyxin (G; human). (H-P) C-Terminal fusion constructs (linker amino acid length indicated by the numbers): annexin (A4)-12-TagRFP-T (H; human; illustrated with ionomycin-induced translocation to the plasma and nuclear membranes); lamin B1-10-TagRFP-T (I; human); vinculin-23-TagRFP-T (J; human); clathrin light chain-15-TagRFP-T (K; human); β-actin-7-TagRFP-T (L; human); PTS1-2-TagRFP-T (M; peroximal targeting signal 1); RhoB-15-TagRFP-T (N; human RhoB GTPase with an N-Terminal c-Myc epitope tag; endosome targeting); farnesyl-5-TagRFP-T (O; 20-amino-acid farnesylation signal from c-Ha-Ras); and β-Tubulin-6-TagRFP-T (P; human). All TagRFP-T fusion vectors were expressed in HeLa (CCL-2) cells. Scale bars, 10 mm.

FIG. 11. Characterization of photostable fluorescent protein variants. (A) arc lamp photobleaching curves for mOrange (orange dotted line), mOrange2 (orange dashed line), and mApple (red solid line), and (B) for TagRFP (dotted line) and TagRFP-T (solid line). All photobleaching curves were measured under continuous illumination without neutral density filters and are plotted as intensity versus normalized total exposure time with an initial emission rate of 1000 photons/s.

FIG. 12. mApple photobleaching at different excitation wavelengths. Widefield photobleaching curves for mApple purified protein under oil with excitation using 568/55 nm (solid line), 540/25 nm (dashed line), or 480/30 nm (dotted line) band pass filters, plotted as intensity versus normalized total exposure time with an initial emission rate of 1000 photons/s per molecule.

FIG. 13. Example of reversible photoswitching curves for mEGFP. For both (a) widefield and (b) confocal imaging, cells expressing histone H2B fused to mEGFP were exposed to constant illumination until measurably bleached, then the cells were then allowed to recover in darkness for approximately 1 minute (indicated by the grey bars), after which time they were re-imaged. The initial fluorescence value fₒ, post-bleach fluorescence f_{b}, and post-recovery fluorescence fᵣ are indicated by the arrows. In this experiment, mEGFP exhibits 45% recovery during widefield imaging and 24% recovery during laser scanning confocal imaging. Note that photobleaching times have not been normalized for differences in excitation intensity.

FIG. 14. Reversible photoswitching of TagRFP, TagRFP-T, and Cerulean during widefield microscopy. (a) A fraction of TagRFP fluorescence recovers after both short and sustained photobleaching. Purified TagRFP was bleached on a microscope at ambient temperatures with xenon arc lamp illumination through a 540/25 nm filter for short (˜2 s) or long intervals as indicated by the bars, and allowed to recover in the dark while fluorescence intensity was measured with 50 ms exposures. (b) A fraction of TagRFP-T fluorescence recovers after short photobleaching, but not after sustained photobleaching. (c) Cerulean demonstrates fluorescence recovery after short (˜10 s) and sustained photobleaching through a 420/20 nm filter. Exposure intervals are indicated by bars. Note that photobleaching times are raw, and have not been adjusted for different illumination powers and the different extinction coefficients and quantum yields as is done to derive normalized photostability measurements.

FIG. 15. High-resolution crystal structure of eqFP611. Cartoon representation of the related fluorescent protein eqFP611, Chain A of PDB ID 1UIS. The chromophore is shown with a stick representation. The amino acid sequence of eqFP611 is given as SEQ ID NO:14.

FIG. 16. Superposition of AvGFP (PDB ID: 1EMA) and DsRed (PDB ID: 1G7K).

### DETAILED DESCRIPTION OF THE INVENTION

The present invention provides novel methods of screening large libraries of fluorescent proteins for enhanced photostability. These novel methods have resulted in the development of a highly photostable monomeric orange variant derived from the fastbleaching mRFP1 derivative mOrange and the identification of a variant of TagRFP with strongly enhanced photostability. The orange variant is 25-fold more photostable than mOrange, twice as photostable as other existing orange monomers, and performed as well as Aequorea GFPs in all fusion constructs tested. The TagRFP variant is 9-fold more photostable than TagRFP while maintaining most of the brightness of the original protein.

It is clear that specific regions proximal to the chromophore of DsRed have a large influence on the modes of photobleaching it is able to undergo. It has been shown that DsRed, when illuminated by a 532 nm pulsed laser, undergoes decarboxylation of Glu215, as well as cis-to-trans isomerization of the chromophore (Habuchi, S. et al., J Am Chem Soc, 127:8977-8984 (2005)). Such chromophore isomerization has been implicated in the photoswitching behavior of kindling fluorescent protein (KFP) (Chudakov, D. M. et al., J Biol Chem, 278:7215-7219 (2003); Andresen, M. et al., Proc Natl Acad Sci USA, 102:13070-13074 (2005)) and Dronpa (Habuchi, S. et al., Proc Natl Acad Sci USA, 102:9511-9516 (2005); Andresen, M. et al., Proc Natl Acad Sci USA (2007)), as well as predecessors to mTFP (Ai, H. W. et al., Biochem J, 400:531-540 (2006); Henderson, J. N. et al., Proc Natl Acad Sci USA, 104:6672-6677 (2007)). It has also previously been observed for Aequorea GFP variants that decarboxylation of the corresponding glutamate (position 222) can lead to changes in optical properties (van Thor, J. J. et al., Nut Struct Biol, 9:37-41 (2002); Bell, A. F. et al., J Am Chem Soc, 125:6919-6926 (2003); van Thor, J. J., Georgiev et al., J Biol Chem, 280:33652-33659 (2005)). However, the observation made in the present invention, that oxidation plays a large role in mOrange photobleaching, suggests that for fast-bleaching proteins such as mOrange and mRFP1, chromophore isomerization and Glu215 decarboxylation may play only a minor role. Additionally, no evidence was found by mass spectrometry that photobleaching using the solar simulator led to any detectable decarboxylation of Glu215 in mOrange.

For mRFP1 variants, the present invention clearly demonstrates the importance of residue 163 in influencing photostability, although context-specific effects of 163 and surrounding residues on different wavelength-shifted variants have also been observed. This region, comprised of residues 64, 97, 99, and 163, appears to be of critical importance in determining the photostability of these monomers. However, of these residues only 163 is in direct contact with the chromophore. It may be that the mutations Q64H and F99Y together lead to a rearrangement of the other side chains in the vicinity of the chromophore, so as to hinder a critical oxidation that leads to loss of fluorescence.

Discrepancies in tubulin and connexin localization when fused to mOrange2 versus mKO or tdTomato can probably be attributed to the three-dimensional structure of the FP and potential steric hindrance in the fusions proteins. mOrange2 contains extended N- and C-Termini derived from EGFP to improve performance in protein fusions, whereas the shorter mKO protein (236 vs. 218 amino acids, respectively) may experience steric interferences that lead to poorer performance in similar fusions. The fused dimeric character of tdTomato effectively doubles the size of this FP, as compared to the monomeric orange FPs, so steric hindrance is the most likely culprit in preventing tubulin localization in these fusions.

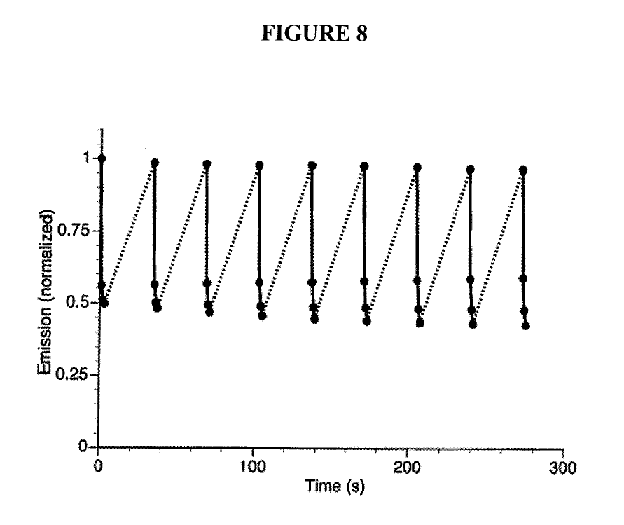

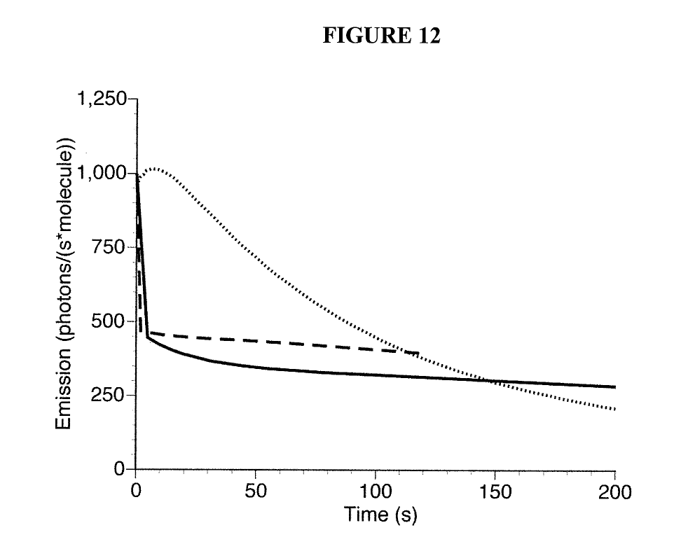

While the present invention does not eliminate unwanted photoswitching behavior of mApple, the variant fluorescent proteins of the invention may be further modified in order to generate further variant that do eliminate this photoswitching behavior. Given that most, if not all, reversibly photoswitchable fluorescent proteins described thus far appear to operate through cis-to-trans isomerization of the chromophore (Andresen et al., PNAS USA, 102(37):13070-4 (2005); Andresen et al., PNAS USA, 104(32):13005-9 (2007); Stiel et al., Biochem J, 402(1) (2007)) it is likely that this mechanism could also be at work in mApple. The fastest-switching mutant of Dronpa, M159T, relaxes in the dark from its temporarily dark state back to fluorescence with a half-Time of 30 sec (Egner et al., Biophys J., 93(9):3285-90 (2007)); mApple is almost completely recovered by 30 sec (FIG. 8), but its behavior is qualitatively similar to Dronpa M159T. Because mApple's spontaneous recovery is already so fast, it can be shown that the initial fast decay of emission is absent with 480 nm excitation (FIG. 12), suggesting that this wavelength stimulates recovery from the dark state as well as the primary fluorescence. Additional investigations of mApple's structure before and after photoswitching may allow the engineering of variants which retain its bright fluorescence but either eliminate (as with mTFP1), or allow controllable photoswitching (as with Dronpa).

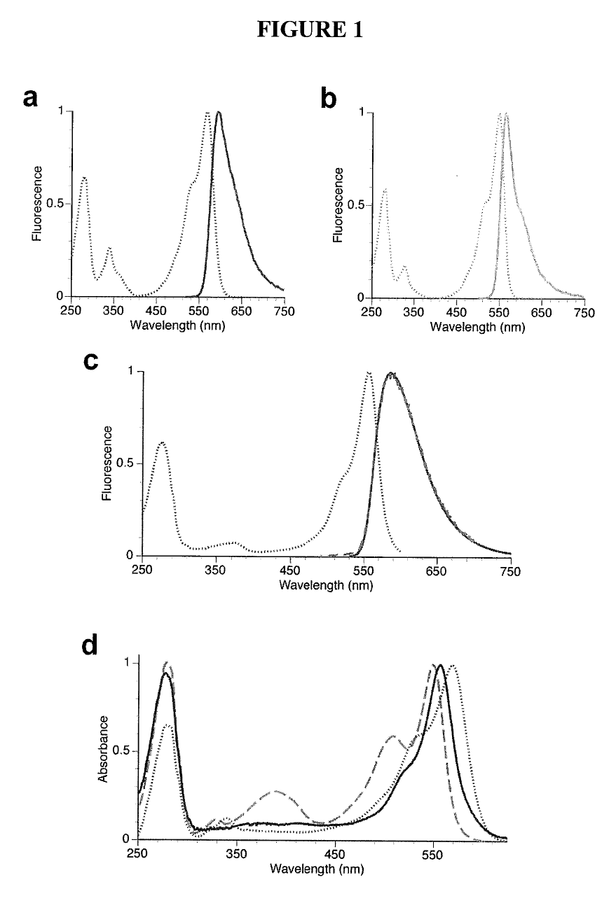

Meanwhile, the existing properties of mApple would seem very attractive for photoactivated localization microscopy with independently running acquisition (PALMIRA) (Egner et al., Biophys J., 93(9):3285-90 (2007)). In this new version of super-resolution microscopy, strong illumination (several kW/cm²) drives most of the fluorophores into a dark state. Individual fluorophores stochastically revert to the fluorescing state, briefly emit a burst of photons, then revert to the dark state. In any one image (whose acquisition time should roughly match the mean duration of an emission burst), the emitters must be sparse enough so that they represent distinct single molecules whose position can be localized to a few nm by centroid-locating algorithms. Superposition of the centroid locations over many images produces a super-resolution composite image. Currently the only genetically encoded, photoreversible fluorophores are Dronpa, asFP595, and their engineered variants. Dronpa fluoresces green and requires an excitation wavelength (488 nm) that slightly stimulates photoactivation of the dark molecules as well as fluorescence and quenching of the bright molecules. asFP595 emits in the red but is very dim (quantum yield <0.001) and tetrameric, whereas mApple also emits red but is quite bright (quantum yield 0.49), very photostable apart from its fast photoswitching, and monomeric. Although FIG. 12 shows photoswitching only down to ˜30% of initial intensity with a few W/cm², PALMIRA operates with up to 3 orders of magnitude higher intensity, so that the activation density may be reducible to <1%. The photoswitching kinetics of the Dronpa mutant favored for PALMIRA, rsFastLime (Dronpa-V157G) (Egner et al., Biophys J., 93(9):3285-90 (2007)) are somewhat different from those of mApple, but specific selection for variants with the desired kinetics or structure-guided design of mutants with altered photoswitching properties should be possible. While laser scanning confocal bleach curves (FIG. 1) suggest that mApple is quite photostable under high intensity intermittent illumination, it is yet to be determined if constant illumination at the higher intensities required for PALMIRA will lead to a larger degree of irreversible photobleaching. Thus, mApple or future variants have the potential to be genetically encoded red FPs complementary to green Dronpa for PALMIRA.

The present invention provides a novel photostability selection method that may be applied to other fluorescent proteins, as demonstrated with TagRFP, which although already contained reasonably good photostability, was still amenable to improvements (see Example 5). From a saturation-mutagenesis library of two chromophore-proximal residues (consisting of 400 independent clones), a single clone was selected with substantially enhanced photostability. While this library size is small compared to the randomly mutagenized libraries used to select mOrange2 as in Example 3, the screening of 400 individual clones manually for photostability would be a resource-intensive and time-consuming proposition in the absence of the novel screening methods of the present invention. Thus, the novel screening methods of the present invention proved highly useful even in this case. The selected mutant, TagRFP-T, should prove to be a very useful addition to the fluorescent protein arsenal, as it is the most photostable monomeric fluorescent protein of any color yet described and possibly the most photostable fluorescent protein yet evaluated, under both arc lamp and confocal laser illumination.

In one embodiment, the present invention provides a novel directed evolution approach for the development of improved fluorescent proteins. The novel fluorescent proteins of the invention, which have been isolated using these methods, are proof that photostability selection can be successfully applied to the development of improved fluorescent proteins, including mFRP1 and other monomeric fluorescent proteins. Starting from the bright but highly photolabile mOrange, the novel methods of the invention have led to the development of the variant, mOrange2, whose photostability is among the highest of any currently available fluorescent proteins, and which is a highly reliable fusion partner. The novel selection methods of the invention have also utilized to identify highly photostable TagRFP variants from a site-directed mutant library. Thus, the novel screening methods of the invention are expected to be applicable to any of the large number of existing fluorescent proteins, and, with modifications, could also be useful in the selection of more efficient photoconvertible and photoswitchable fluorescent proteins (Chudakov, D. M. et al., Nat Biotechnol, 22:1435-1439 (2004); Habuchi, S. et al., Proc Natl Acad Sci USA, 102:9511-9516 (2005); Wiedenmann, J. et al., Proc Natl Acad Sci USA, 101:15905-15910 (2004); Chudakov, D. M. et al., J Biol Chem, 278:7215-7219 (2003); Verkhusha, V. V. and Sorkin, A., Chem Biol, 12:279-285 (2005); Ando, R. et al., Proc Natl Acad Sci USA, 99:12651-12656 (2002); Lukyanov, K. A. et al., Nut Rev Mol Cell Biol (2005); Patterson, G. H. and Lippincott-Schwartz, J., Methods, 32:445-450 (2004); Tsutsui, H. et al., EMBO Rep, 6:233-238 (2005)). Potential enhancements to these selection methods may include, for example, time-lapse imaging of bacterial plates during bleaching in order to enable direct selection for the longest bleaching half-time (independent of absolute brightness) and the use of higher intensity illumination from other light sources (such as lasers) during screening to select against non-linear photobleaching behavior.

In one embodiment, the present invention provides novel fluorescent protein variants with increased photostability. The novel fluorescent protein variants provided by the invention are generally variants of known fluorescent proteins, for example AvGFP, DsRed, mRFP1, mOrange, mCherry, eqFP611, eqFP578, and the like, which have been mutated or evolved in order to achieve a greater photostability. A variant fluorescent protein having increased photostability may otherwise have identical or highly similar spectral properties to the reference fluorescent protein from which it was derived. Alternatively, a photostable fluorescent protein may be a spectral variant or have altered spectral properties with respect to the parent fluorescent protein from which it was derived.

In a specific embodiment, the present invention provides fluorescent protein variants of eqFP578, which are more photostable than TagRFP. In one embodiment, the present invention provides a photostable fluorescent protein comprising an amino acid substitution of Thr at a residue corresponding to residue 158 in a fluorescent protein derived from eqFP578 (SEQ ID NO:18). In one embodiment, the fluorescent protein derived from eqFP578 is TagRFP or TurboRFP. In certain embodiments, the invention provides a photostable fluorescent protein of SEQ ID NO:1, comprising an S158T mutation. In other embodiments, a photostable fluorescent protein comprising an S158T mutation has an amino acid sequence that is at least about 85%, 90%, or 95% identical to SEQ ID NO:1. In one embodiment of the invention, the fluorescent protein derived from eqFP578 further comprises GFP-like sequences at the N-Terminus, C-Terminus, or both. In a particular embodiment, the photostable fluorescent protein is TagRFP-T0.1 (SEQ ID NO:13) or TagRFP-T (SEQ ID NO:7), or has an amino acid sequence that is at least about 85%, 90%, or 95% identical to SEQ ID NO:13 or SEQ ID NO:7, wherein the protein is more photostable than TagRFP (SEQ ID NO:1). In one embodiment, the invention provides an isolated polypeptide of SEQ ID NO:1 comprising the following mutation: S158T.

In another specific embodiment, the present invention provides fluorescent protein variants of DsRed (SEQ ID NO:5) having increased photostability as compared to DsRed. In one embodiment, the present invention provides a photostable fluorescent protein comprising an amino acid substitution of Tyr at a residue corresponding to residue 99 in a fluorescent protein derived from DsRed (SEQ ID NO:5). In a particular embodiment, the fluorescent protein is derived from mRFP1 (SEQ ID NO:12), or any of the mFruits (U.S. Pat. No. 7,157,566; Shu et al., Biochemistry, 45(32):9639-9647 (2006); U.S. patent application Ser. No. 10/931,304 published as U.S. 20050196768; Shaner et al., Nature Methods, 2(12):905-9 (2005)), including without limitation, mCherry, mRaspberry, mPlum, mBanana, mOrange, mApple, mStrawberry, mGrape, mHoneydew, and mTangerine. In a specific embodiment, a photostable protein provided by the invention is derived from mOrange.

In one particular embodiment, the present invention provides a photostable fluorescent protein variant of mRFP1 (SEQ ID NO:12), comprising an F99Y mutation or a Q64H/F99Y double mutation. In another embodiment, a photostable fluorescent protein of the invention has an amino acid sequence that is at least about 85%, 90%, or 95% identical to SEQ ID NO:12, further comprising an F99Y mutation or a Q64H/F99Y double mutation. In one embodiment, the present invention comprises a photostable fluorescent protein variant of mOrange (SEQ ID NO:3), comprising an F99Y mutation or a Q64H/F99Y double mutation. In certain embodiments, the fluorescent protein has an amino acid sequence that is at least about 85%, 90%, or 95% identical to SEQ ID NO:3, further comprising an F99Y mutation or a Q64H/F99Y double mutation.

In another embodiment of the invention, a fluorescent protein having increased photostability relative to mRFP1 (SEQ ID NO:12), may further comprise an E160K mutation, a G196D mutation, or both. In one particular embodiment, the present invention provides a fluorescent protein variant having a sequence substantially identical to SEQ ID NO:3, comprising an F99Y mutation or a Q64H/F99Y double mutation, and further comprising at least one mutation selected from E160K and G196D. In one embodiment, the variant fluorescent protein comprises the mutations G64H, F99Y, E160K, and G196D. In certain embodiments, the fluorescent protein has an amino acid sequence that is at least about 85%, 90%, or 95% identical to SEQ ID NO:3, wherein said protein is more photostable than mOrange (SEQ ID NO:3). In a particular embodiment, the photostable fluorescent protein comprises a sequence substantially identical to mOrange2 (SEQ ID NO:11). In one embodiment, the invention provides an isolated polypeptide of SEQ ID NO:3, comprising the following mutations: G64H, F99Y, E160K, and G196D.

In another particular embodiment, the present invention provides a fluorescent protein, mApple0.5, having a polypeptide sequence substantially identical to SEQ ID NO:3, comprising the mutations: G40A, T66M, A71V, A73V, V104I, V105I, T106H, T108N, E117V, G159S, M163K, T174A, and G196D. In another embodiment, the invention provides a fluorescent protein, mApple, having a polypeptide sequence substantially identical to SEQ ID NO:3, comprising the mutations: R17H, G40A, T66M, A71V, A73I, K92R, V104I, V105I, T106H, T108N, E117V, S147E, G159S, M163K, T174A, S175A, G196D, and T202V. In one embodiment, the invention provides an isolated polypeptide of SEQ ID NO:3, comprising the following mutations: R17H, G40A, T66M, A71V, A73I, K92R, V104I, V105I, T106H, T108N, E117V, S147E, G159S, M163K, T174A, S175A, G196D, and T202V.

In one embodiment of the invention, fluorescent protein variants of the invention may be useful for fluorescent resonance energy transfer (FRET). In certain embodiments, the present invention provides a polypeptide probe suitable for use in fluorescence resonance energy transfer (FRET), comprising at least one fluorescent protein variant of the invention.

In another embodiment of the invention, nucleic acids are provided that encode for fluorescent proteins of the invention. In certain embodiments, the nucleic acids of the invention encode for fluorescent protein variants that have increased photostability with respect to a parent or reference fluorescent protein. Nucleic acids encoding any of the fluorescent proteins described herein are embraced by the present invention. Also provided by the present invention are vectors comprising the nucleic acids of the inventions. In certain embodiments, the nucleic acids of the invention may be functionally linked to a regulatory control element, such as a promoter or enhancer sequence.

In one particular embodiment, the invention provides a nucleic acid that encodes a fluorescent protein variant of eqFP578 or eqFP611, wherein said variant has a greater photostability as compared to the parent fluorescent protein. In a certain embodiment, nucleic acids of the invention encode fluorescent protein variants of TagRFP, TagRFP-T, or TurboRFP. In certain embodiments, a nucleic acid of the invention encodes a fluorescent protein variant having an amino acid sequence that is at least about 85%, 90%, or 95% identical to SEQ ID NO:1, SEQ ID NO:7, or SEQ ID NO:19 comprising an S158T mutation, wherein said variant protein has increased photostability with respect to the parent fluorescent protein. In a particular embodiment of the invention, an isolated nucleic acid comprising a sequence encoding a polypeptide of SEQ ID NO:1 comprising an S158T mutation is provided. In another specific embodiment, the nucleic acid encodes for a polypeptide of SEQ ID NO:7. In yet another embodiment, the invention provides a vector comprising a nucleic acid encoding a fluorescent protein variant of eqFP611, eqFP578, TagRFP, TagRFP-T, or TurboRFP.

In another embodiment, the invention provides a nucleic acid that encodes a fluorescent protein variant of DsRed, mRFP1, mOrange, or mCherry, wherein said variant has a greater photostability as compared to the parent fluorescent protein. In a certain embodiment, the invention provides a nucleic acid encoding a fluorescent protein variant having an amino acid sequence that is at least about 85%, 90%, or 95% identical to SEQ ID NO:5, SEQ ID NO:12, SEQ ID NO:3, or SEQ ID NO:6, comprising an F99Y mutation, wherein said variant has increased photostability with respect to the parent fluorescent protein. In a related embodiment, a polypeptide encoded by a nucleic acid of the invention further comprises a Q64H mutation. In one embodiment, the invention provides a nucleic acid encoding a fluorescent protein variant having an amino acid sequence that is at least about 85%, 90%, or 95% identical to SEQ ID NO:5, SEQ ID NO:12, SEQ ID NO:3, or SEQ ID NO:6 comprising a Q64H/F99Y double mutation, wherein said variant has increased photostability with respect to the parent fluorescent protein. In yet other embodiments, the polypeptide encoded by a nucleic acid of the invention may further comprise at least one residue selected from a Lys at residue 160 and an Asp at residue 196. In a particular embodiment, the invention provides an isolated nucleic acid encoding a polypeptide that is substantially identical to SEQ ID NO:5, SEQ ID NO:12, SEQ ID NO:3, or SEQ ID NO:6 comprising the following mutations: G64H, F99Y, E160K, and G196D. In one embodiment, a nucleic acid of the invention encodes for a polypeptide of SEQ ID NO:11. In yet another embodiment, the invention provides a vector comprising a nucleic acid encoding a fluorescent protein variant of DsRed, mRFP1, mOrange, or mCherry.

In yet another embodiment, the invention provides a nucleic acid encoding a fluorescent protein variant of mOrange comprising the following mutations: G40A, T66M, A71V, A73V, V104I, V105I, T106H, T108N, E117V, G159S, M163K, T174A, and G196D. In another embodiment, the invention provides a nucleic acid encoding a fluorescent protein variant of mOrange comprising the following mutations: R17H, G40A, T66M, A71V, A73I, K92R, V104I, V105I, T106H, T108N, E117V, S147E, G159S, M163K, T174A, S175A, G196D, and T202V. In certain embodiments of the invention, the variant of mOrange has an amino acid sequence that is at least about 85%, 90%, or 95% identical to SEQ ID NO:3. In another embodiment, the invention provides a nucleic acid encoding a polypeptide of SEQ ID NO:9 or SEQ ID NO:10. In yet another embodiment, the invention provides a nucleic acid encoding a fluorescent protein variant of mOrange, wherein the variant displays reversible photoswitching behavior. Vectors comprising nucleic acids encoding fluorescent variants of mOrange are also provided herein.

In one embodiment, the invention provides reversibly photoswitching fluorescent protein variants. In certain embodiments, the invention provides variants of DsRed that display reversible photoswitching behavior. In a particular embodiment, the photoswitching variants comprise an amino acid sequence that is at least about 85%, 90%, or 95% identical to SEQ ID NO:3, wherein said variant displays reversible photoswitching behavior. In one embodiment, the reversibly photoswitching fluorescent protein variants of the invention comprise an amino acid sequence that is at least about 85%, 90%, or 95% identical to mApple0.5 (SEQ ID NO:9) or mApple (SEQ ID NO:10). In yet other embodiments, the variants have an amino acid sequence of SEQ ID NO:9 or SEQ ID NO:10. In certain embodiments of the invention, the photoswitching fluorescent protein variants are useful for nanoscale spatial resolution spectroscopy, “nanoscopy” (for a review, see Hell SW., Science 2007 May 25; 316(5828):1153-8; and Peters R., Nanomed. 2008 February; 3(1):1-4) or photoactivated localization microscopy with independently running acquisition (PALMIRA).

The invention further provides vectors containing polynucleotides, and host cell comprising a polynucleotide or vector. Also provided are recombinant nucleic acid molecules, which include at least one polynucleotide encoding a fluorescent protein variant operatively linked to one or more other polynucleotides. The one or more other polynucleotides can be, for example, a transcription regulatory element such as a promoter or polyadenylation signal sequence, or a translation regulatory element such as a ribosome binding site. Such a recombinant nucleic acid molecule can be contained in a vector, which can be an expression vector, and the nucleic acid molecule or the vector can be contained in a host cell.

The vector generally contains elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCOBRL, Gaithersburg Md.) or can be constructed by one skilled in the art (see, for example, Meth. Enzymol., Vol. 185, Goeddel, ed. (Academic Press, Inc., (1990)); Jolly, Canc. Gene Ther., 1:51-64 (1994); Flotte, J, Bioenerg. Biomemb., 25:37-42 (1993); Kirshenbaum et al., J. Clin. Invest., 92:381-387 (1993)); each of which is incorporated herein by reference).

A vector for containing a polynucleotide encoding a fluorescent protein variant can be a cloning vector or an expression vector, and can be a plasmid vector, viral vector, and the like. Generally, the vector contains a selectable marker independent of that encoded by a polynucleotide of the invention, and further can contain transcription or translation regulatory elements, including a promoter sequence, which can provide tissue specific expression of a polynucleotide operatively linked thereto, which can, but need not, be the polynucleotide encoding the fluorescent protein variant, for example, a tandem fluorescent protein, thus providing a means to select a particular cell type from among a mixed population of cells containing the introduced vector and recombinant nucleic acid molecule contained therein.

Where the vector is a viral vector, it can be selected based on its ability to infect one or few specific cell types with relatively high efficiency. For example, the viral vector also can be derived from a virus that infects particular cells of an organism of interest, for example, vertebrate host cells such as mammalian host cells. Viral vectors have been developed for use in particular host systems, particularly mammalian systems and include, for example, retroviral vectors, other lentivirus vectors such as those based on the human immunodeficiency virus (HIV), adenovirus vectors, adeno-associated virus vectors, herpesvirus vectors, vaccinia virus vectors, and the like (see Miller and Rosman, BioTechniques, 7:980-990 (1992); Anderson et al., Nature, 392:25-30 (Suppl.) (1998); Verna and Somia, Nature, 389:239-242 (1997); Wilson, New Engl. J. Med., 334:1185-1187 (1996), each of which is incorporated herein by reference)).

Recombinant production of a fluorescent protein variant, which can be a component of a fusion protein or tandem fluorescent protein, involves expressing a polypeptide encoded by a polynucleotide. A polynucleotide encoding the fluorescent protein variant is a useful starting material. Polynucleotides encoding fluorescent protein are disclosed herein or otherwise known in the art, and can be obtained using routine methods, then can be modified such that the encoded fluorescent protein has improved photostability or reversible photoswitching behavior. For example, a polynucleotide encoding a red fluorescent protein from Discosoma (DsRed) can be isolated by PCR from cDNA of the Discosoma coral, or obtained from a commercially available source (CLONTECH). PCR methods are well known and routine in the art (see, for example, U.S. Pat. No. 4,683,195; Mullis et al., Cold Spring Harbor Symp. Ouant. Biol., 51:263 (1987); Erlich, ed., “PCR Technology” (Stockton Press, NY, 1989)). A variant form of the fluorescent protein then can be made by site-specific mutagenesis of the polynucleotide encoding the fluorescent protein. Similarly, a tandem fluorescent protein can be expressed from a polynucleotide prepared by PCR or obtained otherwise, using primers that can encode, for example, a peptide linker, which operatively links a first monomer and at least a second monomer of a fluorescent protein.

The construction of expression vectors and the expression of a polynucleotide in transfected cells involves the use of molecular cloning techniques also well known in the art (see Sambrook et al., In “Molecular Cloning: A Laboratory Manual” (Cold Spring Harbor Laboratory Press 1989); “Current Protocols in Molecular Biology” (eds., Ausubel et al.; Greene Publishing Associates, Inc., and John Wiley & Sons, Inc. 1990 and supplements). Expression vectors contain expression control sequences operatively linked to a polynucleotide sequence of interest; for example, that encodes a fluorescent protein variant, as indicated above. The expression vector can be adapted for function in prokaryotes or eukaryotes by inclusion of appropriate promoters, replication sequences, markers, and the like. An expression vector can be transfected into a recombinant host cell for expression of fluorescent protein variant, and host cells can be selected, for example, for high levels of expression in order to obtain a large amount of isolated protein. A host cell can be maintained in cell culture, or can be a cell in vivo in an organism. A fluorescent protein variant can be produced by expression from a polynucleotide encoding the protein in a host cell such as E. coli. Discosoma DsRed-related fluorescent proteins, for example, are best expressed by cells cultured between about 15° C. and 30° C., although higher temperatures such as 37° C. can be used. After synthesis, the fluorescent proteins are stable at higher temperatures and can be used in assays at such temperatures.

In another embodiment, the present invention provides tandem fluorescent proteins, comprising two fluorescent proteins operatively linked by a peptide linker. In a particular embodiment, the tandem fluorescent protein comprises a single polypeptide sequence. In certain embodiments, a tandem fluorescent protein of the invention comprises at least one fluorescent protein variant having increased photostability with respect to a parent or reference fluorescent protein. In certain embodiments, the at least one photostable fluorescent protein variant is derived from a fluorescent protein selected from DsRed, eqFP611, and eqFP578. In particular embodiments, the at least one fluorescent protein is a variant of TurboRFP, TagRFP, mRFP1, mOrange, or mCherry, wherein said variant comprises increased photostability as compared to the parent fluorescent protein. In some embodiments, a tandem fluorescent protein may comprise a fluorescent protein that has not been engineered for increased photostability. In one embodiment of the invention, tandem fluorescent proteins comprising two photostable fluorescent protein variants are provided. Tandem fluorescent proteins may comprise two of the same fluorescent protein or two different fluorescent proteins.

In certain embodiments of the invention a peptide linker can be of variable length, where, for example, the peptide linker is about 10 to about 25 amino acids long, or about 12 to about 22 amino acids long. In some embodiments, the peptide linker is selected from GHGTGSTGSGSS (SEQ ID NO:20), RMGSTSGSTKGQL (SEQ ID NO:21), and RMGSTSGSGKPGSGEGSTKGQL (SEQ ID NO:22). Generally, the peptide linkers embraced by the present invention are not fluorescent, and thus do not interfere with either intermolecular or intramolecular FRET performed by the fluorescent proteins of the invention. Many suitable peptide linkers are known in the art, for example, see U.S. Pat. Nos. 7,332,598, 6,852,849, and 6,803,188. In certain embodiments of the invention, the peptide linker may comprise a protease recognition site or an ion binding site. In particular embodiments of the invention, the cleavage of the linker sequence by a protease, or the binding of the linker by an ion may result in a measurable change in a fluorescent property of the tandem fluorescent protein. In this fashion, the photostable tandem fluorescent proteins of the invention may be used to detect enzyme activity or ion concentration.

In one embodiment, the invention provides tandem fluorescent protein variants that are competent for fluorescence resonance energy transfer (FRET), comprising at least one photostable fluorescent protein variant of the invention. Tandem fluorescent proteins may be competent for intermolecular FRET and/or intramolecular FRET. Tandem fluorescent proteins of the invention that are competent for FRET may comprise one photostable fluorescent protein variant or two fluorescent protein variants.

The present invention also provides fusion proteins comprising any protein of interest operatively joined to at least one fluorescent protein variant of the invention. This fusion protein can optionally contain a peptide tag. For example, a polyhistidine tag containing, for example, six histidine residues, can be incorporated at the N-Terminus or C-Terminus of the fluorescent protein variant, which then can be isolated in a single step using nickel-chelate chromatography. Additional peptide tags, including a GST tag, a c-myc peptide, a FLAG epitope, or any ligand (or cognate receptor), including any peptide epitope (or antibody, or antigen binding fragment thereof, that specifically binds the epitope are well known in the art and similarly can be used. (see, for example, Hopp et al., Biotechnology, 6:1204 (1988); U.S. Pat. No. 5,011,912, each of which is incorporated herein by reference). In certain embodiments, the fluorescent protein variant is derived from a fluorescent protein selected from DsRed, eqFP611, and eqFP578. In particular embodiments, the fluorescent protein is a variant of TurboRFP, TagRFP, mRFP1, mOrange, or mCherry, wherein said variant comprises increased photostability as compared to the parent fluorescent protein.

The present invention also provides nucleic acids encoding any of the tandem fluorescent proteins or fusion fluorescent proteins of the invention. Additionally, vectors comprising tandem fluorescent proteins or fusion fluorescent proteins of the present invention are also provided herewith. In an additional embodiment, the present invention provides methods of expressing a fluorescent protein variant of the present invention using a nucleic acid encoding said variant protein.

In yet other embodiments, the invention provides kits comprising at least one fluorescent protein variant of the invention. Similarly, the invention provides kits comprising at least one polynucleotide encoding a fluorescent protein variant of the invention. In some embodiments, a kit of the invention may comprise at least one fluorescent protein variant of the invention and at least one polynucleotide encoding a fluorescent protein variant of the invention.

In one embodiment, the present invention provides host cells comprising a fluorescent protein variant or polynucleotide encoding a fluorescent protein variant, tandem fluorescent protein, or fusion fluorescent protein of the invention. Suitable host cells include, without limitation, bacteria, yeasts, fungi, and animal and plant cells. Non-limiting examples of suitable prokaryotic host cells include a strain of E. coli, a strain of Enterobacter, a strain of Salmonella, a strain of Bacilli, such as B. subtilis or B. licheniformis, a strain of Pseudomonas, a strain of Streptomyces, and the like. Non-limiting examples of eukaryotic host cells include without limitation, a yeast, such as Saccharomyces cerevisiae, Schizosaccharomyces pombe, or a Kluyveromyces yeast, Neurospora crassa, a fungus or mold, such as Neurospora, Penicillium, Tolypocladium, Aspergillus, an insect cell, such as a Drosophilai cell or an Anopheles cell, a mammalian cell, such as a CHO cell, a COS cell, a human cell, a 293 cell, a HeLa cell, a Hep G2 cell, a mouse cell, and the like.

In one embodiment, the present invention provides methods of detecting the expression of a protein using a fluorescent protein variant or a nucleic acid encoding a fluorescent variant protein of the invention. In one embodiment, the method comprises the steps of expressing a fusion protein comprising a fluorescent protein variant of the invention and a target protein of interest in a cell or cellular extract, and detecting the fluorescence of said fluorescent protein variant, thereby detecting the expression of a target protein of interest. In another embodiment, the method comprises the steps of expressing a fusion protein comprising a fluorescent protein variant of the invention and a peptide or protein that binds to a target protein of interest in a cell or a cellular extract, and detecting a difference in fluorescence or a property related to fluorescence, such as relative or total fluorescence, fluorescence anisotropy or fluorescence polarization, thereby detecting expression of a target protein of interest.

In another embodiment, the present invention provides a method of detecting a protein-protein interaction using a fluorescent protein variant or a nucleic acid encoding a fluorescent variant protein of the invention. In a particular embodiment, the method comprises the steps of contacting a fusion protein comprising a fluorescent protein variant of the invention and a first protein of interest with a second protein of interest, and detecting a change in fluorescence or a change in a property related to fluorescence, thereby detecting an interaction between a first protein of interest and a second protein of interest. In one embodiment, the second protein of interest comprises a fusion protein of said second protein of interest and a fluorescent protein. In certain embodiments, said second fluorescent protein may be a fluorescent protein variant of the invention. Protein-protein interactions may be detected by measuring FRET between two suitable fluorescent proteins, by measuring relative fluorescence, by measuring fluorescence anisotropy, by measuring fluorescence polarization, or by any other well known method in the art. Protein-protein interactions may be measured in vivo, in vitro, ex vivo, in a cell, in a cellular extract, and the like.

In another embodiment, the present invention provides a method of detecting the localization of a protein using a fluorescent protein variant or a nucleic acid encoding a fluorescent variant protein of the invention. In one embodiment, the method comprises the steps of expressing a fusion protein comprising a fluorescent protein variant of the invention and a target protein of interest in a cell, detecting the fluorescence of said fluorescent protein variant, and determining the cellular location of said fluorescence, thereby determining the localization of said target protein of interest.

In yet another embodiment of the invention, a method of detecting protein motility using a fluorescent protein variant or a nucleic acid encoding a fluorescent variant protein of the invention is provided. In one embodiment, the method comprises the steps of expressing a fusion protein comprising a fluorescent protein variant of the invention and a target protein of interest in a cell, performing time-sequential observations of fluorescence in said cell, and detecting differences in fluorescence between said time-sequential observation, thereby detecting protein motility of a target protein of interest. Methods of determining the cellular localization of a target protein of interest and the dynamics thereof are well known in the art, and can be found, for example, in Campbell and Ashe, Methods Enzymol., 431:33-45 (2007).

In one embodiment of the invention, a method for evolving a photostable fluorescent protein variant is provided. Any fluorescent protein known in the art may be evolved with the methods of the present invention to increase its photostability, including without limitation, AvGFP, DsRed, eqFP578, eqFP611, and spectral variants thereof, such as mOrange, mRFP1, TagRFP, TurboRFP, YFP, CFP, and the like. Generally, the methods of evolving photostable fluorescent proteins comprise iterative mutagenesis followed by selection for photostability. These steps may be repeated as necessary until a fluorescent protein containing the desired photostability properties is isolated. In certain embodiments, the methods of the invention comprise the steps of mutating a nucleic acid encoding a fluorescent protein and performing a selection assay for a mutated fluorescent protein with increased photostability as compared to the parent fluorescent protein. These steps may optionally be repeated one or more times. One of skill will be able to determine the optimal time and conditions for protein photobleaching, which will depend upon properties of the parent fluorescent protein being evolved and the desired level of photostability. A multitude of light sources may be used to photobleach the variant fluorescent proteins of the invention, for example, a solar simulator, an arc-lamp, a laser, and the like.

In one specific embodiment, the invention provides a photostability selection assay comprising the steps of photobleaching a plurality of colonies or clones, containing mutated nucleic acids encoding for a fluorescent protein for which increased photostability is desired, for a predetermined time and selecting the brightest post-bleach clones. In certain embodiments, the method may further comprise the steps of determining the brightness of a fluorescent protein variant or colony expressing a fluorescent protein variant prior to photobleaching. In yet another embodiment, the method may further comprise calculating the photobleaching half-life of one or more fluorescent protein variants. In yet other embodiments of the invention, the method employed may further include screening for other desired fluorescent properties, such as a desired excitation peak and/or emission peak, a desired quantum yield, a desired extinction coefficient, a desired maturation time, a desired pKa, a desired sensitivity to pH or ion concentration, and the like. Photostable fluorescent proteins generated by the methods of the present invention are provides herewith.

In yet another embodiment, the invention provides a method of evolving or generating a fluorescent protein variant that displays reversible photoswitching behavior. In a particular embodiment, the method comprises mutating a plurality of nucleic acids encoding a parent fluorescent protein, and then screening the resultant mutant proteins for photoswitching behavior. In certain embodiments, the screening method entails successive rounds of photobleaching for a predetermined time followed by a predetermined recovery time. After a given number of photobleaching/recovery steps the fluorescent proteins may then be screened for fluorescence properties equal to those prior to photobleaching.

### DEFINITIONS

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The term “nucleic acid molecule” or “polynucleotide” refers to a deoxyribonucleotide or ribonucleotide polymer in either single-stranded or double-stranded form, and, unless specifically indicated otherwise, encompasses polynucleotides containing known analogs of naturally occurring nucleotides that can function in a similar manner as naturally occurring nucleotides. It will be understood that when a nucleic acid molecule is represented by a DNA sequence, this also includes RNA molecules having the corresponding RNA sequence in which “U” (uridine) replaces “T” (thymidine).

The term “recombinant nucleic acid molecule” refers to a non-naturally occurring nucleic acid molecule containing two or more linked polynucleotide sequences. A recombinant nucleic acid molecule can be produced by recombination methods, particularly genetic engineering techniques, or can be produced by a chemical synthesis method. A recombinant nucleic acid molecule can encode a fusion protein, for example, a fluorescent protein variant of the invention linked to a polypeptide of interest. The term “recombinant host cell” refers to a cell that contains a recombinant nucleic acid molecule. As such, a recombinant host cell can express a polypeptide from a “gene” that is not found within the native (non-recombinant) form of the cell.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, mRNA, oligonucleotide, and polynucleotide.

Reference to a polynucleotide “encoding” a polypeptide means that, upon transcription of the polynucleotide and translation of the mRNA produced therefrom, a polypeptide is produced. The encoding polynucleotide is considered to include both the coding strand, whose nucleotide sequence is identical to an mRNA, as well as its complementary strand. It will be recognized that such an encoding polynucleotide is considered to include degenerate nucleotide sequences, which encode the same amino acid residues. Nucleotide sequences encoding a polypeptide can include polynucleotides containing introns as well as the encoding exons.

The term “expression control sequence” refers to a nucleotide sequence that regulates the transcription or translation of a polynucleotide or the localization of a polypeptide to which to which it is operatively linked. Expression control sequences are “operatively linked” when the expression control sequence controls or regulates the transcription and, as appropriate, translation of the nucleotide sequence (i.e., a transcription or translation regulatory element, respectively), or localization of an encoded polypeptide to a specific compartment of a cell. Thus, an expression control sequence can be a promoter, enhancer, transcription terminator, a start codon (ATG), a splicing signal for intron excision and maintenance of the correct reading frame, a STOP codon, a ribosome binding site, or a sequence that targets a polypeptide to a particular location, for example, a cell compartmentalization signal, which can target a polypeptide to the cytosol, nucleus, plasma membrane, endoplasmic reticulum, mitochondrial membrane or matrix, chloroplast membrane or lumen, medial trans-Golgi cisternae, or a lysosome or endosome. Cell compartmentalization domains are well known in the art and include, for example, a peptide containing amino acid residues 1 to 81 of human type II membrane-anchored protein galactosyltransferase, or amino acid residues 1 to 12 of the presequence of subunit IV of cytochrome c oxidase (see also, Hancock et al., EMBO J., 10:4033-4039 (1991); Buss et al., Mol. Cell. Biol., 8:3960-3963 (1988); U.S. Pat. No. 5,776,689, each of which is incorporated herein by reference).

The term “polypeptide” or “protein” refers to a polymer of two or more amino acid residues. The terms apply to amino acid polymers in which one or more amino acid residue is an artificial chemical analogue of a corresponding naturally occurring amino acid, as well as to naturally occurring amino acid polymers. The term “recombinant protein” refers to a protein that is produced by expression of a nucleotide sequence encoding the amino acid sequence of the protein from a recombinant DNA molecule.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. One of skill in the art will also recognize that conservative substitutions to a protein embraced by the present invention will be well tolerated, especially at residues whose side chains are surface exposed, residues located in the loop regions that connect individual β-strands in the β-barrel fold of fluorescent proteins, and in residues distal to the chromophore, or whose side chains do not contribute to the electron environment of the chromophore. Additionally, one of skill in the art will recognize that non-conservative mutations in fluorescent proteins are well tolerated in residues whose side chains are solvent exposed (Lawrence et al., J Am Chem. Soc., 129(33):10110-2 (2007)).

The following six groups each contain amino acids that are conservative substitutions for one another: 1) Alanine (Ala, A), Serine (Ser, S), Threonine (Thr, T); 2) Aspartic acid (Asp, D), Glutamic acid (Glu, E); 3) Asparagine (Asn, N), Glutamine (Gln, Q); 4) Arginine (Arg, R), Lysine (Lys, K); 5) Isoleucine (Ile, I), Leucine (Leu, L), Methionine (Met, M), Valine (Val, V); and 6) Phenylalanine (Phe, F), Tyrosine (Tyr, Y), Tryptophan (Trp, V).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/, or the like). Such sequences are then said to be “substantially identical” or “substantially similar.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100, 200, 300, 400, 500, or more amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. In certain embodiments, a comparison window may be at least about 25, 50, 75, 100, 150, 200, 250, 300, 400, 500, 600, or more positions. Methods of alignment of sequences for comparison are well-known in the art. Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Nat'l. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds., Wiley Interscience (1987-2005)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

A subject nucleotide sequence is considered “substantially complementary” to a reference nucleotide sequence if the complement of the subject nucleotide sequence is substantially identical to the reference nucleotide sequence. The term “stringent conditions” refers to a temperature and ionic conditions used in a nucleic acid hybridization reaction. Stringent conditions are sequence dependent and are different under different environmental parameters. Generally, stringent conditions are selected to be about 5° C. to 20° C. lower than the thermal melting point (Tm) for the specific sequence at a defined ionic strength and pH. The Tm is the temperature, under defined ionic strength and pH, at which 50% of the target sequence hybridizes to a perfectly matched probe.

The term “allelic variants” refers to polymorphic forms of a gene at a particular genetic locus, as well as cDNAs derived from mRNA transcripts of the genes, and the polypeptides encoded by them. The term “preferred mammalian codon” refers to the subset of codons from among the set of codons encoding an amino acid that are most frequently used in proteins expressed in mammalian cells as chosen from the following list: Gly (GGC; GGG); Glu (GAG); Asp (GAC); Val (GUG, GUC); Ala (GCC, GCU); Ser (AGC, UCC); Lys (AAG); Asn (AAC); Met (AUG); Ile (AUC); Thr (ACC); Trp (UGG); Cys (UGC); Tyr (UAU, UAC); Leu (CUG); Phe (UUC); Arg (CGC, AGG, AGA); Gln (CAG); His (CAC); and Pro (CCC).

The term “operatively linked” or “operably linked” or “operatively joined” or the like, when used to describe “chimeric” or “fusion” proteins, refer to polypeptide sequences that are placed in a physical and functional relationship to each other. In an embodiment, the functions of the polypeptide components of the chimeric molecule are unchanged compared to the functional activities of the parts in isolation. For example, a fluorescent protein of the present invention can be fused to a polypeptide of interest. In this case, it is preferable that the fusion molecule retains its fluorescence, and the polypeptide of interest retains its original biological activity. In some embodiments of the present invention, the activities of either the fluorescent protein or the protein of interest can be reduced relative to their activities in isolation. Such fusions can also find use with the present invention.

In another embodiment, a “tandem fluorescent protein” variant of the invention comprises two “operatively linked” fluorescent protein units or moieties. The two units are linked in such a way that each maintains its fluorescence activity. The first and second units in the tandem fluorescent protein need not be identical. In certain embodiments of the invention, the two fluorescent protein moieties of a tandem fluorescent protein will be arranged such that they display fluorescent resonance energy transfer (FRET) when the acceptor moiety is excited with light of the appropriate wavelength. In another embodiment, a third polypeptide of interest can be operatively linked to the tandem fluorescent protein, thereby forming a three part fusion protein. In certain embodiments of the invention, two fluorescent protein moieties of a tandem fluorescent protein will be joined or connected with a linker moiety.

Fluorescent molecules are useful in fluorescence resonance energy transfer, FRET, which involves a donor molecule and an acceptor molecule. To optimize the efficiency and detectability of FRET between a donor and acceptor molecule, several factors need to be balanced. The emission spectrum of the donor should overlap as much as possible with the excitation spectrum of the acceptor to maximize the overlap integral. Also, the quantum yield of the donor moiety and the extinction coefficient of the acceptor should be as high as possible to maximize R_{O}, which represents the distance at which energy transfer efficiency is 50%. However, the excitation spectra of the donor and acceptor should overlap as little as possible so that a wavelength region can be found at which the donor can be excited efficiently without directly exciting the acceptor because fluorescence arising from direct excitation of the acceptor can be difficult to distinguish from fluorescence arising from FRET. Similarly, the emission spectra of the donor and acceptor should overlap as little as possible so that the two emissions can be clearly distinguished. High fluorescence quantum yield of the acceptor moiety is desirable if the emission from the acceptor is to be measured either as the sole readout or as part of an emission ratio. One factor to be considered in choosing the donor and acceptor pair is the efficiency of fluorescence resonance energy transfer between them. Preferably, the efficiency of FRET between the donor and acceptor is at least 10%, more preferably at least 50% and even more preferably at least 80%. In certain embodiments, the efficiency of FRET between two fluorescent protein moieties may be at least about 10%, 20%, 30%, 40%, 50%, 60%, 70%, 80%, 90% or higher.

The term “fluorescent property” refers to the molar extinction coefficient at an appropriate excitation wavelength, the fluorescence quantum efficiency, the shape of the excitation spectrum or emission spectrum, the excitation wavelength maximum and emission wavelength maximum, the ratio of excitation amplitudes at two different wavelengths, the ratio of emission amplitudes at two different wavelengths, the excited state lifetime, or the fluorescence anisotropy. A measurable difference in any one of these properties between a wild type fluorescent protein, such as Discosoma sp. DsRed or Aequorea GFP, and a spectral variant, or a mutant thereof, is useful. A measurable difference can be determined by determining the amount of any quantitative fluorescent property, e.g., the amount of fluorescence at a particular wavelength or the integral of fluorescence over the emission spectrum. Determining ratios of excitation amplitude or emission amplitude at two different wavelengths (“excitation amplitude ratioing” and “emission amplitude ratioing”, respectively) are particularly advantageous because the ratioing process provides an internal reference and cancels out variations in the absolute brightness of the excitation source, the sensitivity of the detector, and light scattering or quenching by the sample.

As used herein, the term “fluorescent protein” refers to any protein that can fluoresce when excited with an appropriate electromagnetic radiation, except that chemically tagged proteins, wherein the fluorescence is due to the chemical tag, and polypeptides that fluoresce only due to the presence of certain amino acids such as tryptophan or tyrosine, whose emission peaks at ultraviolet wavelengths (i.e., less that about 400 nm) are not considered fluorescent proteins for purposes of the present invention. In general, a fluorescent protein useful for preparing a composition of the invention or for use in a method of the invention is a protein that derives its fluorescence from autocatalytically forming a chromophore. A fluorescent protein can contain amino acid sequences that are naturally occurring or that have been engineered (i.e., variants or mutants). When used in reference to a fluorescent protein, the term “mutant” or “variant” refers to a protein that is different from a reference protein. For example, a spectral variant of Discosoma sp. DsRed can be derived from the naturally occurring DsRed by engineering mutations such as amino acid substitutions into the reference DsRed protein. For example mApple and mOrange2 are spectral variants of DsRed that contains substitutions with respect to DsRed. Similarly, TagRFP is a spectral variant of EqRFP, which contains amino acid substitutions with respect to EqRFP. Non-limiting examples of fluorescent protein well suited for use with the present invention include DsRed, AvGFP, HcRed, AmCyan, AcGFP, ZsYellow, ZsGreen, EqRFP, AsRed, TagRFP (Merzlyak, E. M. et al., Nat Methods, 4:555-557 (2007)), TurboRFP, mutant proteins thereof, and the like. Other useful fluorescent proteins can be found, for example, in Miyawaki (Cell Structure and Function 27:343-7 (2002)), Shaner et al. (Journal of Cell Science, 120(24):4247-60 (2007)), Shaner et al. (Nature Methods, 2(12):905-9 (2005)), and Stepanenko et al. (Curr Protein Pept Sci., 9(4):338-69) (2008)).

Many cnidarians use green fluorescent proteins as energy transfer acceptors in bioluminescence. The term “green fluorescent protein” is used broadly herein to refer to a protein that fluoresces green light, for example, Aequorea GFP. GFPs have been isolated from the Pacific Northwest jellyfish, Aequorea victoria, the sea pansy, Renilla reniformis, and Phialidium gregarium (Ward et al., Photochem. Photobiol., 35:803-808 (1982); Levine et al., Comp. Biochem. Physiol., 72B:77-85 (1982), each of which is incorporated herein by reference). Similarly, reference is made herein to “red fluorescent proteins”, which fluoresce red, “cyan fluorescent proteins,” which fluoresce cyan, and the like. RFPs, for example, have been isolated from the corallimorph Discosoma (Matz et al., Nature Biotechnology, 17:969-973 (1999)). The term “red fluorescent protein,” or “RFP” is used in the broadest sense and specifically covers the Discosoma RFP (DsRed), and red fluorescent proteins from any other species, such as coral and sea anemone, as well as variants thereof as long as they retain the ability to fluoresce.

A variety of Aequorea GFP-related fluorescent proteins having useful excitation and emission spectra have been engineered by modifying the amino acid sequence of a naturally occurring GFP from A. Victoria (see Prasher et al., Gene, 111:229-233 (1992); Heim et al., Proc. Natl. Acad. Sci. USA, 91:12501-12504 (1994); U.S. Pat. No. 5,625,048; International application PCT/US95/14692, now published PCT WO96/23810; U.S. Pat. No. 7,022,826; U.S. Pat. No. 6,852,849, each of which is incorporated herein by reference).

A variety of Discosoma RFP-related fluorescent proteins having useful excitation and emission spectra have been engineered by modifying the amino acid sequence of a naturally occurring RFP from Discosoma sp. (see Matz et al., Nature Biotechnology, 17:969-73 (1999); U.S. Pat. No. 7,157,566; U.S. Pat. No. 7,329,735; U.S. Pat. No. 7,332,598, each of which is incorporated herein by reference). Discosoma Red-related fluorescent proteins include, for example, wild type (native) DsRed (Matz et al., Nature Biotechnology, 17:969-73 (1999)), allelic variants of SEQ ID NO:5, spectral variants of RFP, such as mRFP1 (U.S. Pat. No. 7,005,511), and the mFruits (U.S. Pat. No. 7,157,566; Shu et al., Biochemistry, 45(32):9639-9647 (2006); U.S. patent application Ser. No. 10/931,304, published as U.S. 20050196768; Shaner et al., Nature Methods, 2(12):905-9 (2005)), dsFP593, and enhanced and otherwise modified forms thereof (U.S. Pat. No. 7,157,566; U.S. Pat. No. 7,329,735; U.S. Pat. No. 7,332,598, each of which is incorporated herein by reference), including RFP-related fluorescent proteins having one or more folding mutations, and fragments of the proteins that are fluorescent, for example, an RFP from which the two N-Terminal amino acid residues have been removed.

As used herein, the numbering of amino acids in DsRed or mRFP1 variants conforms to the wild-Type sequence of DsRed (SEQ ID NO:5), in which residues 66-68 of wild-Type DsRed (Gln-Tyr-Gly) are homologous to the chromophore-forming residues 65-67 of GFP (Ser-Tyr-Gly). When amino acid residues are inserted at or near position 6, they are numbered to preserve the DsRed numbering for the rest of the protein; for example, where ENNMA (SEQ ID NO:16) or EDNMA (SEQ ID NO:17) are inserted at position 6, such as in some of the mFruits, these residues are numbered as residues 6a, 6b, 6c, 6d, and 6e, respectively. For example, an F99Y mutation in mOrange corresponds to a Phe to Tyr mutation in amino acid 104 of SEQ ID NO:3, as numbered in the sequence listing. Similarly, the numbering of amino acids in eqFP578 or eqFP611 mutants conforms to the wild type sequence of eqFP578 (SEQ ID NO:18) or eqFP611 (SEQ ID NO:611).

The term “oligomer” refers to a complex formed by the specific interaction of two or more polypeptides. A “specific interaction” or “specific association” is one that is relatively stable under specified conditions, for example, physiologic conditions. Reference to a “propensity” of proteins to oligomerize indicates that the proteins can form dimers, trimers, tetramers, or the like under specified conditions. Generally, fluorescent proteins such as GFPs and DsRed have a propensity to oligomerize under physiologic conditions although, as disclosed herein, fluorescent proteins also can oligomerize, for example, under pH conditions other than physiologic conditions. The conditions under which fluorescent proteins oligomerize or have a propensity to oligomerize can be determined using well known methods as disclosed herein or otherwise known in the art.

The term “probe” refers to a substance that specifically binds to another substance (a “target”). Probes include, for example, antibodies, polynucleotides, receptors and their ligands, and generally can be labeled so as to provide a means to identify or isolate a molecule to which the probe has specifically bound. The term “label” refers to a composition that is detectable with or without the instrumentation, for example, by visual inspection, spectroscopy, or a photochemical, biochemical, immunochemical or chemical reaction. Useful labels include, for example, phosphorus-32, a fluorescent dye, a fluorescent protein, an electron-dense reagent, an enzyme (such as is commonly used in an ELISA), a small molecule such as biotin, digoxigenin, or other haptens or peptide for which an antiserum or antibody, which can be a monoclonal antibody, is available. It will be recognized that a fluorescent protein variant of the invention, which is itself a detectable protein, can nevertheless be labeled so as to be detectable by a means other than its own fluorescence, for example, by incorporating a radionuclide label or a peptide tag into the protein so as to facilitate, for example, identification of the protein during its expression and isolation of the expressed protein, respectively. A label useful for purposes of the present invention generally generates a measurable signal such as a radioactive signal, fluorescent light, enzyme activity, and the like, either of which can be used, for example, to quantitate the amount of the fluorescent protein variant in a sample.

The term “nucleic acid probe” refers to a polynucleotide that binds to a specific nucleotide sequence or sub-sequence of a second (target) nucleic acid molecule. A nucleic acid probe generally is a polynucleotide that binds to the target nucleic acid molecule through complementary base pairing. It will be understood that a nucleic acid probe can specifically bind a target sequence that has less than complete complementarity with the probe sequence, and that the specificity of binding will depend, in part, upon the stringency of the hybridization conditions. A nucleic acid probe can be labeled as with a radionuclide, a chromophore, a lumiphore, a chromogen, a fluorescent protein, or a small molecule such as biotin, which itself can be bound, for example, by a streptavidin complex, thus providing a means to isolate the probe, including a target nucleic acid molecule specifically bound by the probe. By assaying for the presence or absence of the probe, one can detect the presence or absence of the target sequence or sub-sequence. The term “labeled nucleic acid probe” refers to a nucleic acid probe that is bound, either directly or through a linker molecule, and covalently or through a stable non-covalent bond such as an ionic, van der Waals or hydrogen bond, to a label such that the presence of the probe can be identified by detecting the presence of the label bound to the probe.

The term “isolated” or “purified” refers to a material, such as a protein or nucleic acid, that is substantially or essentially free from components that normally accompany the material in its native state in nature. Purity or homogeneity generally are determined using analytical chemistry techniques such as polyacrylamide gel electrophoresis, high performance liquid chromatography, and the like. A polynucleotide or a polypeptide is considered to be isolated when it is the predominant species present in a preparation. Generally, an isolated protein or nucleic acid molecule represents greater than 80% of the macromolecular species present in a preparation, often represents greater than 90% of all macromolecular species present, usually represents greater than 95%, of the macromolecular species, and, in particular, is a polypeptide or polynucleotide that purified to essential homogeneity such that it is the only species detected when examined using conventional methods for determining purity of such a molecule.

As used herein, the term “photostability” refers to a measure of a fluorescent protein's resistance to the loss of fluorescence upon extended excitation. Typically, the photostability of a fluorescent protein will be expressed in terms of the photobleaching half-life of the protein, e.g., the time it takes to achieve 50% photobleaching in a homogenous sample of a fluorescent protein. One standard measure of photostability is the time for bleaching a fluorescent protein from an initial emission rate of 1,000 photons per second down to an emission rate of 500 photons per second (See, Shaner et al., Nature Methods, 2(12):905-9 (2005)). As such, a fluorescent protein variant is considered to have “increased photostability” if said variant has a longer photobleaching half-life as compared to a reference or wild-Type fluorescent protein. Fluorescent protein variants having increased photostability may also be referred to herein as “photostable fluorescent protein variants”, “photostable fluorescent proteins”, and the like. Typically, with respect to a variant fluorescent protein, a reference or wild-Type fluorescent protein is a fluorescent protein from which said variant is derived, mutated, or evolved from. For example, DsRed is a reference fluorescent protein with respect to mApple. mOrange is also a reference protein with respect to mApple. Generally, a variant fluorescent protein with increased photostability will have a photobleaching half-life that is at least about 10% greater than a reference or wild-Type fluorescent protein. In certain embodiments, a variant fluorescent protein with increased photostability may have a photobleaching half-life that is at least about 5% greater, or at least about 10%, 15%, 20%, 25%, 30%, 40%, 50%, 60%, 70%, 80%, 90%, or 100%, greater as compared to the photobleaching half-life of a wild-Type or reference fluorescent protein. In other embodiments, a variant fluorescent protein having increased photostability may have a photobleaching half-life that is at least about 1-fold, 2-fold, 3-fold, 4-fold, 5-fold, 6-fold, 7-fold, 8-fold, 9-fold, 10-fold, or more greater than the photobleaching half-life of a wild-Type or reference fluorescent protein.

### Kits of the Invention

The present invention also provides kits to facilitate and/or standardize use of compositions provided by the present invention, as well as facilitate the methods of the present invention. Materials and reagents to carry out these various methods can be provided in kits to facilitate execution of the methods. As used herein, the term “kit” is used in reference to a combination of articles that facilitate a process, assay, analysis or manipulation.

Kits can contain chemical reagents (e.g., polypeptides or polynucleotides) as well as other components. In addition, kits of the present invention can also include, for example but not limited to, apparatus and reagents for sample collection and/or purification, apparatus and reagents for product collection and/or purification, reagents for bacterial cell transformation, reagents for eukaryotic cell transfection, previously transformed or transfected host cells, sample tubes, holders, trays, racks, dishes, plates, instructions to the kit user, solutions, buffers or other chemical reagents, suitable samples to be used for standardization, normalization, and/or control samples. Kits of the present invention can also be packaged for convenient storage and safe shipping, for example, in a box having a lid.

In some embodiments, for example, kits of the present invention can provide a fluorescent protein variant of the invention, a polynucleotide vector (e.g., a plasmid) encoding a fluorescent protein variant of the invention, bacterial cell strains suitable for propagating the vector, and reagents for purification of expressed fusion proteins. Alternatively, a kit of the present invention can provide the reagents necessary to conduct mutagenesis of a fluorescent protein in order to generate a protein variant having increased photostability or reversible photoswitching behavior.

A kit can contain one or more compositions of the invention, for example, one or a plurality of fluorescent protein variants, which can be a portion of a fusion protein, or one or a plurality of polynucleotides that encode the polypeptides. The fluorescent protein variant can be a mutated fluorescent protein having increased photostability, or can be a tandem fluorescent protein variant or fusion fluorescent protein variant and, where the kit comprises a plurality of fluorescent protein variants, the plurality can be a plurality of the mutated fluorescent protein variants, or of the tandem fluorescent proteins or fusion fluorescent protein variants, or a combination thereof.

A kit of the invention also can contain one or a plurality of recombinant nucleic acid molecules, which encode fluorescent protein variants, which can be the same or different, and can further include, for example, an operatively linked second polynucleotide containing or encoding a restriction endonuclease recognition site or a recombinase recognition site, or any polypeptide of interest. In addition, the kit can contain instructions for using the components of the kit, particularly the compositions of the invention that are contained in the kit.

Such kits can be particularly useful where they provide a plurality of different fluorescent protein variants because the artisan can conveniently select one or more proteins having the fluorescent properties desired for a particular, application. Similarly, a kit containing a plurality of polynucleotides encoding different fluorescent protein variants provides numerous advantages. For example, the polynucleotides can be engineered to contain convenient restriction endonuclease or recombinase recognition sites, thus facilitating operative linkage of the polynucleotide to a regulatory element or to a polynucleotide encoding a polypeptide of interest or, if desired, for operatively linking two or more the polynucleotides encoding the fluorescent protein variants to each other.

### Uses of Fluorescent Protein Variants

A fluorescent protein variant of the invention is useful in any method that employs a fluorescent protein. Thus, the fluorescent protein variants, including photostable fluorescent proteins and fluorescent proteins having reversible photoswitching behavior, are useful as fluorescent markers in the many ways fluorescent markers already are used, including, for example, coupling fluorescent protein variants to antibodies, polynucleotides or other receptors for use in detection assays such as immunoassays or hybridization assays, or to track the movement of proteins in cells. For intracellular tracking studies, a first (or other) polynucleotide encoding the fluorescent protein variant is fused to a second (or other) polynucleotide encoding a protein of interest and the construct, if desired, can be inserted into an expression vector. Upon expression inside the cell, the protein of interest can be localized based on fluorescence, without concern that localization of the protein is an artifact caused by oligomerization of the fluorescent protein component of the fusion protein. In one embodiment of this method, two proteins of interest independently are fused with two fluorescent protein variants that have different fluorescent characteristics.

The fluorescent protein variants of this invention are useful in systems to detect induction of transcription. For example, a nucleotide sequence encoding a photostable fluorescent proteins or a fluorescent protein having reversible photoswitching behavior can be fused to a promoter or other expression control sequence of interest, which can be contained in an expression vector, the construct can be transfected into a cell, and induction of the promoter (or other regulatory element) can be measured by detecting the presence or amount of fluorescence, thereby allowing a means to observe the responsiveness of a signaling pathway from receptor to promoter.

A fluorescent protein variant of the invention also is useful in applications involving FRET, which can detect events as a function of the movement of fluorescent donors and acceptors towards or away from each other. One or both of the donor/acceptor pair can be a fluorescent protein variant of the invention. Such a donor/acceptor pair provides a wide separation between the excitation and emission peaks of the donor, and provides good overlap between the donor emission spectrum and the acceptor excitation spectrum. One of skill in the art will be able to select appropriate donor and acceptor fluorescent proteins for use in FRET (Hanson and Hanson, Comb Chem High Throughput Screen, 11(7):505-13 (2008); Shaner et al., Nat Methods, 2(12):905-9 (2005)).

FRET can be used to detect cleavage of a substrate having the donor and acceptor coupled to the substrate on opposite sides of the cleavage site. Upon cleavage of the substrate, the donor/acceptor pair physically separate, eliminating FRET. Such an assay can be performed, for example, by contacting the substrate with a sample, and determining a qualitative or quantitative change in FRET (see, e.g., U.S. Pat. No. 5,741,657, which is incorporated herein by reference). A fluorescent protein variant donor/acceptor pair also can be part of a fusion protein coupled by a peptide having a proteolytic cleavage site (see, e.g., U.S. Pat. No. 5,981,200, which is incorporated herein by reference). FRET also can be used to detect changes in potential across a membrane. For example, a donor and acceptor can be placed on opposite sides of a membrane such that one translates across the membrane in response to a voltage change, thereby producing a measurable FRET (see, e.g., U.S. Pat. No. 5,661,035, which is incorporated herein by reference).

In other embodiments, a fluorescent protein variant of the invention is useful for making fluorescent sensors for protein kinase and phosphatase activities or indicators for small ions and molecules such as Ca²⁺, Zn²⁺, cyclic 3′,5′-adenosine monophosphate, and cyclic 3′,5′-guanosine monophosphate.

Fluorescence in a sample generally is measured using a fluorimeter, wherein excitation radiation from an excitation source having a first wavelength, passes through excitation optics, which cause the excitation radiation to excite the sample. In response, a fluorescent protein variant in the sample emits radiation having a wavelength that is different from the excitation wavelength. Collection optics then collect the emission from the sample. The device can include a temperature controller to maintain the sample at a specific temperature while it is being scanned, and can have a multi-axis translation stage, which moves a microtiter plate holding a plurality of samples in order to position different wells to be exposed. The multi-axis translation stage, temperature controller, autofocusing feature, and electronics associated with imaging and data collection can be managed by an appropriately programmed digital computer, which also can transform the data collected during the assay into another format for presentation. This process can be miniaturized and automated to enable screening many thousands of compounds in a high throughput format. These and other methods of performing assays on fluorescent materials are well known in the art (see, e.g., Lakowicz, “Principles of Fluorescence Spectroscopy” (Plenum Press 1983); Herman, Meth. Cell Biol., 30:219-243 (1989); Turro, “Modern Molecular Photochemistry” (Benjamin/Cummings Publ. Co., Inc., 1978), pp. 296-361, each of which is incorporated herein by reference).

Accordingly, the present invention provides a method for identifying the presence of a molecule in a sample. Such a method can be performed, for example, by linking a fluorescent protein variant of the invention to the molecule, and detecting fluorescence due to the fluorescent protein variant in a sample suspected of containing the molecule. The molecule to be detected can be a polypeptide, a polynucleotide, or any other molecule, including, for example, an antibody, an enzyme, or a receptor, and the fluorescent protein variant can be a tandem fluorescent protein.

The sample to be examined can be any sample, including a biological sample, an environmental sample, or any other sample for which it is desired to determine whether a particular molecule is present therein. Preferably, the sample includes a cell or an extract thereof. The cell can be obtained from a vertebrate, including a mammal such as a human, or from an invertebrate, and can be a cell from a plant or an animal. The cell can be obtained from a culture of such cells, for example, a cell line, or can be isolated from an organism. As such, the cell can be contained in a tissue sample, which can be obtained from an organism by any means commonly used to obtain a tissue sample, for example, by biopsy of a human. Where the method is performed using an intact living cell or a freshly isolated tissue or organ sample, the presence of a molecule of interest in living cells can be identified, thus providing a means to determine, for example, the intracellular compartmentalization of the molecule.

A fluorescent protein variant can be linked to the molecule directly or indirectly, using any linkage that is stable under the conditions to which the protein-molecule complex is to be exposed. Thus, the fluorescent protein and molecule can be linked via a chemical reaction between reactive groups present on the protein and molecule, or the linkage can be mediated by linker moiety, which contains reactive groups specific for the fluorescent protein and the molecule. It will be recognized that the appropriate conditions for linking the fluorescent protein variant and the molecule are selected depending, for example, on the chemical nature of the molecule and the type of linkage desired. Where the molecule of interest is a polypeptide, a convenient means for linking a fluorescent protein variant and the molecule is by expressing them as a fusion protein from a recombinant nucleic acid molecule, which comprises a polynucleotide encoding, for example, a tandem fluorescent protein operatively linked to a polynucleotide encoding the polypeptide molecule.

A method of identifying an agent or condition that regulates the activity of an expression control sequence also is provided. Such a method can be performed, for example, by exposing a recombinant nucleic acid molecule, which includes a polynucleotide encoding a fluorescent protein variant operatively linked to an expression control sequence, to an agent or condition suspected of being able to regulate expression of a polynucleotide from the expression control sequence, and detecting fluorescence of the fluorescent protein variant due to such exposure. Such a method is useful, for example, for identifying chemical or biological agents, including cellular proteins, that can regulate expression from the expression control sequence, including cellular factors involved in the tissue specific expression from the regulatory element. As such, the expression control sequence can be a transcription regulatory element such as a promoter, enhancer, silencer, intron splicing recognition site, polyadenylation site, or the like; or a translation regulatory element such as a ribosome binding site.

The fluorescent protein variants of the invention also are useful in a method of identifying a specific interaction of a first molecule and a second molecule. Such a method can be performed, for example, by contacting the first molecule, which is linked to a donor first fluorescent protein, and the second molecule, which is linked to an acceptor second fluorescent protein, under conditions that allow a specific interaction of the first molecule and second molecule; exciting the donor; and detecting fluorescence or luminescence resonance energy transfer from the donor to the acceptor, thereby identifying a specific interaction of the first molecule and the second molecule. The conditions for such an interaction can be any conditions under which is expected or suspected that the molecules can specifically interact. In particular, where the molecules to be examined are cellular molecules, the conditions generally are physiological conditions. As such, the method can be performed in vitro using conditions of buffer, pH, ionic strength, and the like, that mimic physiological conditions, or the method can be performed in a cell or using a cell extract.

Luminescence resonance energy transfer entails energy transfer from a chemiluminescent, bioluminescent, lanthanide, or transition metal donor to the variant fluorescent protein moiety. The longer wavelengths of excitation of red fluorescent proteins permit energy transfer from a greater variety of donors and over greater distances than possible with green fluorescent protein variants. Also, the longer wavelengths of emission is more efficiently detected by solid-state photodetectors and is particularly valuable for in vivo applications where red light penetrates tissue far better than shorter wavelengths. Chemiluminescent donors include but are not limited to luminol derivatives and peroxyoxalate systems. Bioluminescent donors include but are not limited to aequorin, obelin, firefly luciferase, Renilla luciferase, bacterial luciferase, and variants thereof. Lanthanide donors include but are not limited to terbium chelates containing ultraviolet-absorbing sensitizer chromophores linked to multiple liganding groups to shield the metal ion from solvent water. Transition metal donors include but are not limited to ruthenium and osmium chelates of oligopyridine ligands. Chemiluminescent and bioluminescent donors need no excitation light but are energized by addition of substrates, whereas the metal-based systems need excitation light but offer longer excited state lifetimes, facilitating time-gated detection to discriminate against unwanted background fluorescence and scattering.

The first and second molecules can be cellular proteins that are being investigated to determine whether the proteins specifically interact, or to confirm such an interaction. Such first and second cellular proteins can be the same, where they are being examined, for example, for an ability to oligomerize, or they can be different where the proteins are being examined as specific binding partners involved, for example, in an intracellular pathway. The first and second molecules also can be a polynucleotide and a polypeptide, for example, a polynucleotide known or to be examined for transcription regulatory element activity and a polypeptide known or being tested for transcription factor activity. For example, the first molecule can comprise a plurality of nucleotide sequences, which can be random or can be variants of a known sequence, that are to be tested for transcription regulatory element activity, and the second molecule can be a transcription factor, such a method being useful for identifying novel transcription regulatory elements having desirable activities.

The present invention also provides a method for determining whether a sample contains an enzyme. Such a method can be performed, for example, by contacting a sample with a tandem fluorescent protein variant of the invention; exciting the donor, and determining a fluorescence property in the sample, wherein the presence of an enzyme in the sample results in a change in the degree of fluorescence resonance energy transfer. Similarly, the present invention relates to a method for determining the activity of an enzyme in a cell. Such a method can be performed, for example, providing a cell that expresses a tandem fluorescent protein variant construct, wherein the peptide linker moiety comprises a cleavage recognition amino acid sequence specific for the enzyme coupling the donor and the acceptor; exciting said donor, and determining the degree of fluorescence resonance energy transfer in the cell, wherein the presence of enzyme activity in the cell results in a change in the degree of fluorescence resonance energy transfer.

Also provided is a method for determining the pH of a sample. Such a method can be performed, for example, by contacting the sample with a first fluorescent protein variant, which can be a tandem fluorescent protein, wherein the emission intensity of the first fluorescent protein variant changes as pH varies between pH 5 and pH 10; exciting the indicator; and determining the intensity of light emitted by the first fluorescent protein variant at a first wavelength, wherein the emission intensity of the first fluorescent protein variant indicates the pH of the sample. It will be recognized that fluorescent protein variants provided by the present invention are useful, either alone or in combination, for the variously disclosed methods of the invention.

The sample used in a method for determining the pH of a sample can be any sample, including, for example, a biological tissue sample, or a cell or a fraction thereof. In addition, the method can further include contacting the sample with a second fluorescent protein variant, wherein the emission intensity of the second fluorescent protein variant changes as pH varies from 5 to 10, and wherein the second fluorescent protein variant emits at a second wavelength that is distinct from the first wavelength; exciting the second fluorescent protein variant; determining the intensity of light emitted by the second fluorescent protein variant at the second wavelength; and comparing the fluorescence at the second wavelength to the fluorescence at the first wavelength. The first (or second) fluorescent protein variant can include a targeting sequence, for example, a cell compartmentalization domain such a domain that targets the fluorescent protein variant in a cell to the cytosol, the endoplasmic reticulum, the mitochondrial matrix, the chloroplast lumen, the medial trans-Golgi cisternae, a lumen of a lysosome, or a lumen of an endosome. For example, the cell compartmentalization domain can include amino acid residues 1 to 81 of human type II membrane-anchored protein galactosyltransferase, or amino acid residues to 12 of the presequence of subunit IV of cytochrome c oxidase.

Other uses of the fluorescent protein variants of the invention will be known to one of skill in the art. Non-limiting examples of these uses can be found, for example, in Stepanenko et al., Curr Protein Pept Sci., 9(4):338-69 (2008); Hanson and Hanson, Comb Chem High Throughput Screen, 11 (7):505-13 (2008); and Shaner et al., Nat Methods, 2(12):905-9 (2005).

### EXAMPLES

### Example 1

Photostability assay and rationale. To simulate illumination conditions on a typical epifluorescence microscope setup, a solar simulator was used, which produces a collimated beam of light approximately 10 cm in diameter from a 1600 W mercury arc lamp. This illumination intensity, while approximately 100- to 200-fold lower than that produced by arc lamp illumination on a microscope without neutral density filters, is sufficient to photobleach the highly photolabile fluorescent protein mOrange to 50% initial intensity after approximately 10 minutes. This reasonably short time for photobleaching entire plates of bacteria expressing fluorescent proteins allowed us to quickly screen libraries of up to 100,000 clones. Heating of plates was minimized by using a cold mirror to eliminate infrared light from the solar simulator beam and by placing the bacteria plates in a custom-built water-cooled aluminum block. At wavelengths necessary to photobleach orange and red fluorescent proteins, we found no substantial decrease in bacterial viability after as long as 2 hours of continuous illumination.

The first attempt to create photostable mRFP1-derived fluorescent proteins began with an analysis of the most photostable existing variant, mCherry. mCherry exhibits very similar excitation and emission spectra to mRFP1, but has improved maturation efficiency and over 10-fold greater photostability as judged by the photon dose required for 50% bleaching. By gathering photobleaching curves for intermediate mutants produced during mCherry directed evolution, it was determined that the M163Q mutation present in mCherry was responsible for its increased photostability. Residue 163 sits immediately adjacent to the chromophore phenolate, and is occupied by a lysine in wild-Type DsRed that forms a salt bridge with the chromophore (Yarbrough, D. et al., Proc Natl Acad Sci USA, 98:462-467 (2001)).

### Example 2

Evolution of a brighter photostable red monomer—mApple. Simultaneously evolution of a brighter and more photostable red fluorescent monomer was undertaken. The relatively photostable variant mCherry exhibits red fluorescence (ex. 587 nm, em. 610 nm) with a pKa of <4.5 and a quantum yield of 0.22. However, it was observed that at very high pH this variant undergoes a transition to a higher-quantum yield (about 0.50) blue-shifted (ex. 568 nm, em. 592 nm) form with a pKa of about 9.5. Since a similar pH-dependence was observed in the early stages of the evolution of mOrange², it was reasoned that restoring threonine 66 in the chromophore of mOrange to the wild-Type glutamine, as in DsRed, (thus restoring red fluorescence) might allow us to find a high-quantum yield red fluorescent variant with a pKa in a practical range.

As predicted, the mOrange T66Q mutant exhibited red fluorescence similar to mCherry, but with a pKa for transition to high-quantum yield red fluorescence at a lower value than mCherry (about 8.0). One round of directed evolution led to the first low-pKa bright red mutant, mApple0.1 (mOrange G40A, T66Q), which had a pKa of 6.4. This mutant, however, exhibited rapid photobleaching and had a substantial fraction of “dead-end” green chromophore which was brightly fluorescent. Subsequent rounds of directed evolution led to the introduction of the mutation M163K, which simultaneously increased photostability markedly and led to almost complete red chromophore maturation. With each round of directed evolution, both photostability screening and brightness screening was included, so that this increase of photostability was maintained with each generation.

After 5 rounds of directed evolution, the variant, mApple0.5 (SEQ ID NO:9), contains 13 mutations relative to mOrange (SEQ ID NO:3) and 17 mutations relative to mCherry (SEQ ID NO:6). After 6 rounds of directed evolution, the final variant, mApple (SEQ ID NO:10), possesses 18 mutations relative to mOrange and 19 mutations relative to mCherry. With a quantum yield of 0.49 and extinction coefficient of 75,000 M⁻¹·cm⁻¹, mApple is more than twice as bright as mCherry. Its reasonably fast maturation time of approximately 30 minutes should additionally allow rapid detection when expressed in cells (see FIG. 1a for spectra, Table 1 for detailed properties, and Table 2 for mutations relative to mOrange).

When subjected to constant illumination, mApple undergoes a small amount of photoactivation, and also displays unusual reversible photoswitching behavior. This photoswitching leads to a reduction in fluorescence emission of between 50 and 70% after several seconds of illumination at typical fluorescence microscope intensities of 1 to 10 W/cm² (e.g., FIG. 1c, a photobleaching curve taken without neutral density filters). For the immediate precursor variant, mApple0.5, this decrease in emission reverses fully within 30 seconds when illumination is discontinued, and cycles of photoswitching and full reversal appear to be repeatable indefinitely (FIG. 8). Because of this photoswitching behavior, mApple displays a short photobleaching t_{1/2} of 4.8 seconds in a standard photobleaching assay (see Table 1). More fortunately, however, Apple appears far more photostable under laser scanning confocal illumination, with a photobleaching tin superior to mOrange and mKO, and approaching that of mCherry (see Table 1 and FIG. 8).

The key difference between the two illumination conditions may be that laser scanning excitation is intermittent for any given pixel, giving time for some recovery in the dark. Also, unless extreme care is taken not to minimize excitation before taking the first image, it is easy to miss the very fast initial phase of decaying emission. All attempts to eliminate mApple's photoswitching behavior by mutagenesis of residues surrounding the chromophore produced unwanted reductions in quantum yield and/or maturation efficiency. However, such photoswitching may make mApple useful for revolutionary new optical techniques for nanoscale spatial resolution (“nanoscopy”).

All reversibly photoswitchable fluorescent proteins described thus far operate through cis-to-trans isomerization of the chromophore (Andresen, M. et al., Proc Natl Acad Sci USA (2007); Stiel, A. C. et al., Biochem J. 402:35-42 (2007), so this mechanism is probably responsible for the photoswitching of mApple. The fastest-switching mutant of Dronpa, M159T, relaxes in the dark from its temporarily dark state back to fluorescence with a half-Time of 30 sec (Egner, A. et al., Biophys J, 93:3285-3290 (2007)); mApple is almost completely recovered by 30 sec (FIG. 8), but its behavior is qualitatively similar to Dronpa M159T. Because mApple's spontaneous recovery is already so fast, systematic exploration of acceleration by short-wavelength illumination has not yet been fully explored, however, the initial fast decay of emission is absent with 480 nm excitation (FIG. 12), suggesting that this wavelength stimulates recovery from the dark state as well as the primary fluorescence.

TABLE 1
Optical properties of novel photostable variants compared with other common monomeric fluorescent proteins.

| | Excitation | Emission | Extinction | Fluorescence | | | to_{1/2} for | | to_{1/2} bleach | to_{1/2} bleach | to_{1/2} bleach |
|---|---|---|---|---|---|---|---|---|---|---|---|
| Fluorescent | maximum | maximum | coefficient | quantum | | | maturation | | (arc lamp)^{b} | (O₂-free)^{c} | (confocal)^{d} |
| protein | (nm) | (nm) | (M⁻¹ · cm⁻¹) | yield | Brightness^{a} | pKa | at 37° C. | | (s) | (s) | (s) |
| mRFP1 | 584 | 607 | 50,000 | 0.25 | 13 | 4.5 | <1 | h | 8.7 | ND^{e} | 210 |
| mCherry | 587 | 610 | 72,000 | 0.22 | 16 | <4.5 | 15 | min. | 96 | ND | 1800 |
| mOrange | 548 | 562 | 71,000 | 0.69 | 49 | 6.5 | 2.5 | h | 9.0 | 250 | 460 |
| DsRed | 558 | 583 | 75,000 | 0.79 | 59 | 4.7 | 10 | h | 326 | ND | ND |
| tdTomato | 554 | 581 | 138,000 | 0.69 | 95 | 4.7 | 60 | min | 98 | ND | 210 |
| mKO | 548 | 559 | 51,600 | 0.60 | 31 | 5.0 | 4.5 | h | 122 | ND | 930 |
| TagRFP^{f} | 555 | 584 | 98,000 | 0.41 | 40 | 3.1 | 100 | min | 37 | 323 | 550 |
| mEGFP | 488 | 507 | 56,000 | 0.60 | 34 | 6.0 | ND | | 174 | ND | 5000 |
| mOrange2 | 549 | 565 | 58,000 | 0.60 | 35 | 6.5 | 4.5 | h | 228 | 228 | 2900 |
| mApple | 568 | 592 | 75,000 | 0.49 | 37 | 6.5 | 30 | min | 4.8 | ND | 1300 |
| TagRFP-T | 555 | 584 | 81,000 | 0.41 | 33 | 4.6 | 100 | min | 337 | >>600 | 6900 |

^{a}Brightness of fully mature protein, (EC · QY)/1000
^{b}Time(s) to bleach to 50% emission intensity under arc-lamp illumination, at an illumination level that causes each molecule to emit 1000 photons/s initially, as measured in our lab. See ref. 15 for more details.
^{c}With arc lamp illumination, equilibrated under O₂-free conditions.
^{d}Time(s) to bleach to 50% emission intensity measured during laser scanning confocal microscopy, at an average illumination level over the scanned area that causes each molecule to emit an average 1000 photons/s initially, as measured in our lab. A 543 nm laser line was used for all proteins except mEGFP, which was bleached with a 488 nm laser.
^{e}ND, not determined.
^{f}All measurements were performed in our lab.

TABLE 2
Mutations of new photostable fluorescent protein variants.

Protein

| | |
|---|---|
| | Mutations relative to mOrange¹ |
| mApple0.1 | G40A/T66Q |
| (SEQ ID | |
| NO: 8) | |
| mApple0.5 | G40A/T66M/A71V/V73I/V104I/V105I/T106H/T108N/ |
| (SEQ ID | E117V/G159S/M163K/T174A/G196D |
| NO: 9) | |
| mApple | R17H/G40A/T66M/A71V/V73I/K92R/V104I/V105I/ |
| (SEQ ID | T106H/T108N/E117V/S147E/G159S/M163K/T174A/ |
| NO: 10) | S175A/G196D/T202V |
| mOrange2 | Q64H/F99Y/E160K/G196D |
| (SEQ ID | |
| NO: 11) | |
| | Mutations relative to TagRFP |
| TagRFP-T | S158T |
| (SEQ ID | |
| NO: 7) | |

¹Amino acid numbers refer to the numbering used in the wtDsRed sequence (SEQ ID NO: 5)

### Example 3

Evolution of a brighter photostable orange monomer—mOrange2. The engineering of a photostable variant of mOrange, which, though it is the brightest of the previously engineered mRFP1 variants, exhibits relatively fast bleaching was undertaken. Because substitutions at position 163 successfully improved photostability during the evolution of both mCherry and mApple, the M163Q mutant of mOrange was initially tested, but it was found that its several-fold enhanced photostability was accompanied by undesirable decreases in quantum yield and maturation efficiency. The M163K mutant of mOrange exhibited substantially enhanced photostability and matured very efficiently, but unfortunately suffered from increased acid sensitivity (pKa −7.0). Because another orange fluorescent protein, mKO (derived from Fungia concinna) (Karasawa, S. et al., Biochem J, 381:307-12 (2004)), is both highly photostable (Shaner, N. C. et al., Nat Methods, 2:905-909 (2005)) and possesses a methionine at the position equivalent to 163 in DsRed, we reasoned that other pathways must exist for increasing photostability in the orange monomer.

To explore alternative photostability-enhancement evolution pathways, iterative random and directed mutagenesis was used in combination with selection using the solar simulator. Initially, a randomly mutagenized library of mOrange was screened by photobleaching on the solar simulator for 15 to 20 minutes per plate and selecting the brightest post-bleach clones. This screen identified a single clone, mOrange F99Y, which had approximately two-fold improved photostability over mOrange (data not shown). Saturation mutagenesis of residue 99 and residues 97 and 163, which potentially have synergistic interactions with residue 99, did not yield further improvements.

A randomly mutagenized library of mOrange F99Y was then constructed, and again screened by photobleaching on the solar simulator, this time increasing illumination time to 40 minutes per plate. This round of screening identified the additional mutation Q64H, which conferred a remarkable 10-fold increase in photostability over the F99Y single mutant. Again, saturation mutagenesis of residues 64, 99, and surrounding residues failed to produce clones that were improved over the original clone identified in the random screen. Additionally, we found that the Q64H mutation alone did not confer substantially enhanced photostability, but rather required the presence of the F99Y mutation. Two further rounds of directed evolution improved the folding efficiency of this variant, resulting in the final clone, mOrange2 (SEQ ID NO:11), which has the additional mutations E160K and G196D.

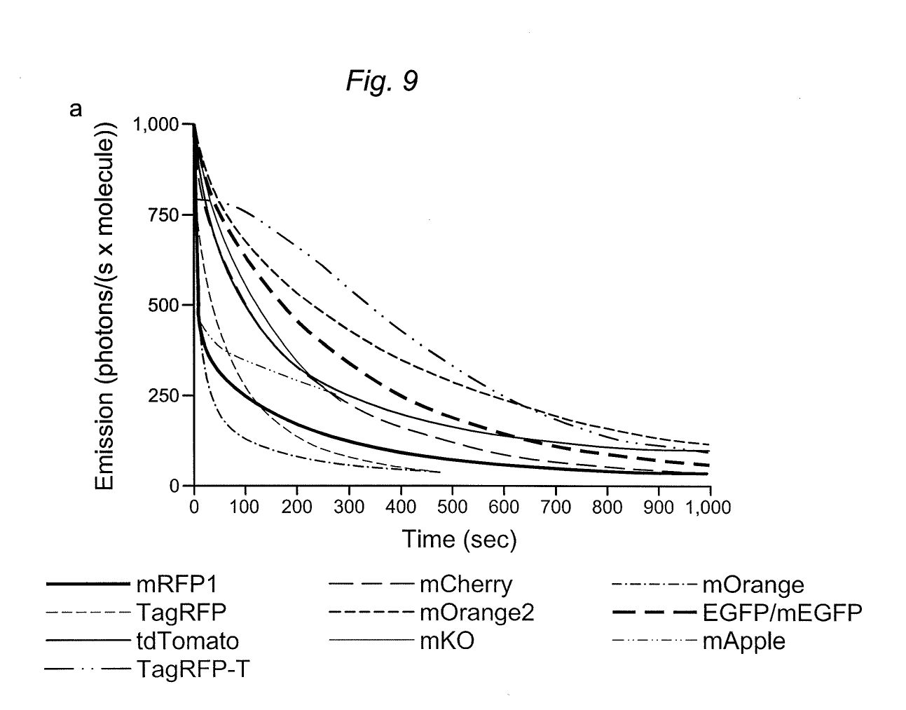

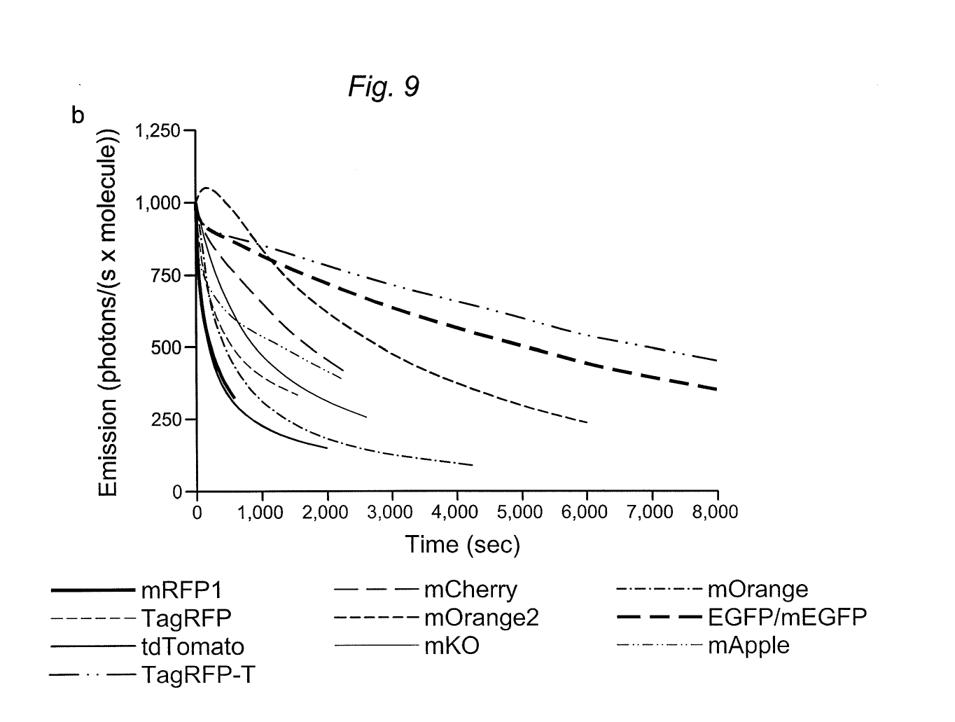

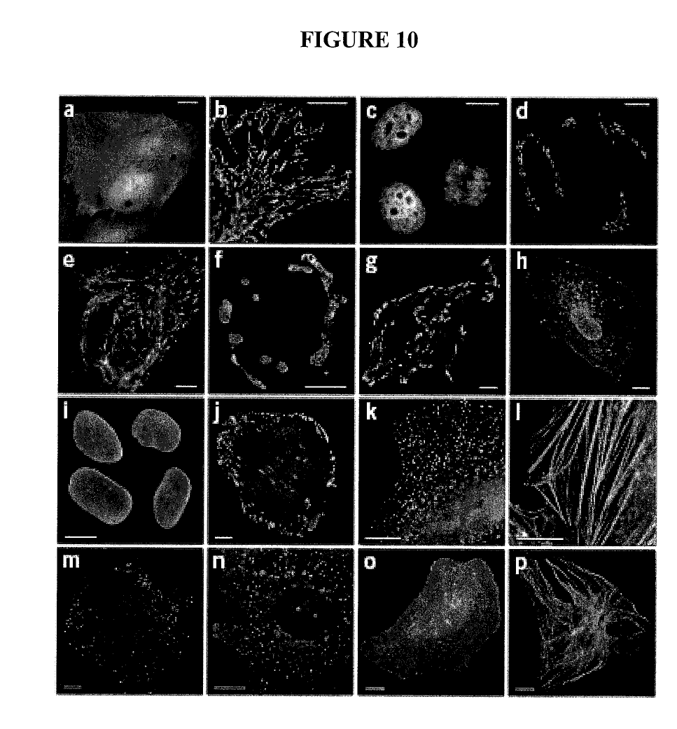

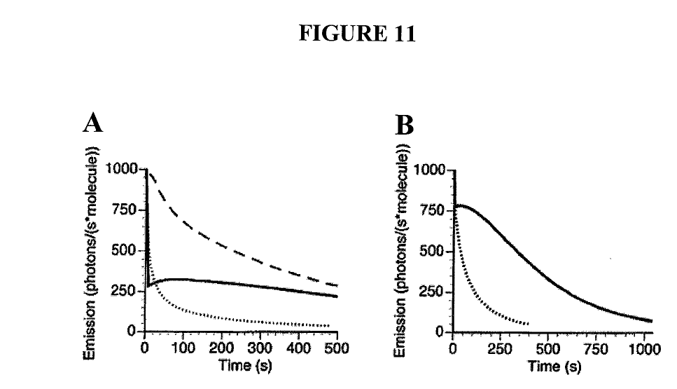

The highly desirable increase in photostability achieved in mOrange2 is balanced by a modest decrease in quantum yield (0.60 versus 0.69) and extinction coefficient (58,000 versus 72,000 M⁻¹·cm⁻¹), together corresponding to a 30% decrease in brightness compared to mOrange. It also exhibits slightly shifted excitation and emission peaks (549 nm and 565 nm) and an increased maturation half-time (4.5 hours versus 2.5 hours). However, its photostability under arc lamp illumination is over 25-fold greater than that of mOrange (FIG. 1c), making it nearly twice as photostable as mKO (Karasawa, S. et al., Biochem J, 381:307-12 (2004)), the previously most photostable known orange monomer (Shaner, N. C. et al., Nat Methods, 2:905-909 (2005)), approximately 6-fold more photostable than TagRFP (Merzlyak, E. M. et al., Nat Methods, 4:555-557 (2007)), a more recent orange-red monomer, and about 1.3-fold more photostable than EGFP (Shaner, N. C. et al., Nat Methods, 2:905-909 (2005)) (see FIG. 1b for spectra, Table 1 for detailed properties, and Table 2 for mutations relative to mOrange). During laser scanning confocal imaging, mOrange2 is approximately 6-fold more photostable than mOrange and 3-fold more photostable than mKO (see FIG. 9). Curiously, the brightness and maturation time of mOrange2 are also quite similar to those for mKO. mOrange2 remains acid-sensitive with a pKa of 6.5, making it undesirable for targeting to acidic compartments, but attractive as a possible marker for exocytosis or other pH-variable processes (Miesenbock, G. et al., Nature, 394:192-195 (1998)). To ensure that the external mutations present in mOrange2 had not increased its propensity to dimerize, we verified its monomeric character using gel filtration.

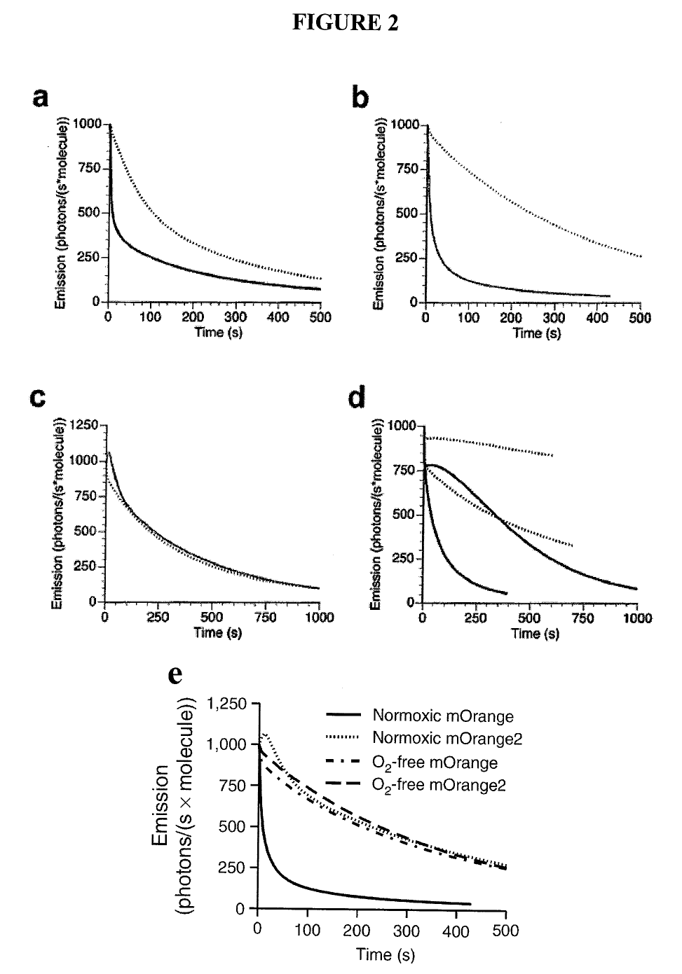

To determine whether the combination of Q64H and F99Y mutations could confer enhanced photostability on related fluorescent protein variants, we introduced these mutations into mRFP1 (SEQ ID NO:12) (U.S. patent application Ser. No. 10/931,304, published as U.S. 2005/0196768), the second-generation variant mCherry (Shaner, N. C. et al., Nat Biotechnol, 22:1567-1572 (2004)), and mApple (described above). As with mOrange, the Q64H mutation alone did not lead to an increase in photostability of any of these variants. However, the combination of Q64H and F99Y conferred an approximately 11-fold increase in photostability to mRFP1, making it as photostable as its successor, mCherry (FIG. 2a). However, these mutations also had undesirable effects on maturation and folding efficiency of mRFP1, making the double mutant suboptimal compared with mCherry. Interestingly, the combination of Q64H and F99Y had no effect on the photostability of mCherry or mApple at all, suggesting that this combination of mutations specifically enhances photostability in mRFP1 variants possessing methionine at position 163. It is tempting to speculate that substitutions at 163 may inhibit photobleaching by the same mechanism as the Q64H/F99Y double mutation.

To determine if photobleaching was occurring through an oxidative mechanism, we measured bleaching curves for mOrange and mOrange2 before and after removing O₂ by equilibration of the bleaching chamber under N₂. Anoxia led to a dramatic increase in mOrange photobleaching half-time (approximately 25-fold, see FIG. 2 and Table I), indicating that the primary mechanism for mOrange photobleaching under normoxic conditions is oxidative. Interestingly, anoxia had almost no effect on the photobleaching curve of mOrange2 (FIG. 2), indicating that its primary bleaching mechanism is fundamentally different from that of mOrange and that the photostability-enhancing mutations almost completely suppress the oxidative bleaching pathway. However, anoxia did prevent the small amount of photoactivation observed for mOrange2 under normal conditions, indicating that this effect remains oxygen-dependent.

### Example 4

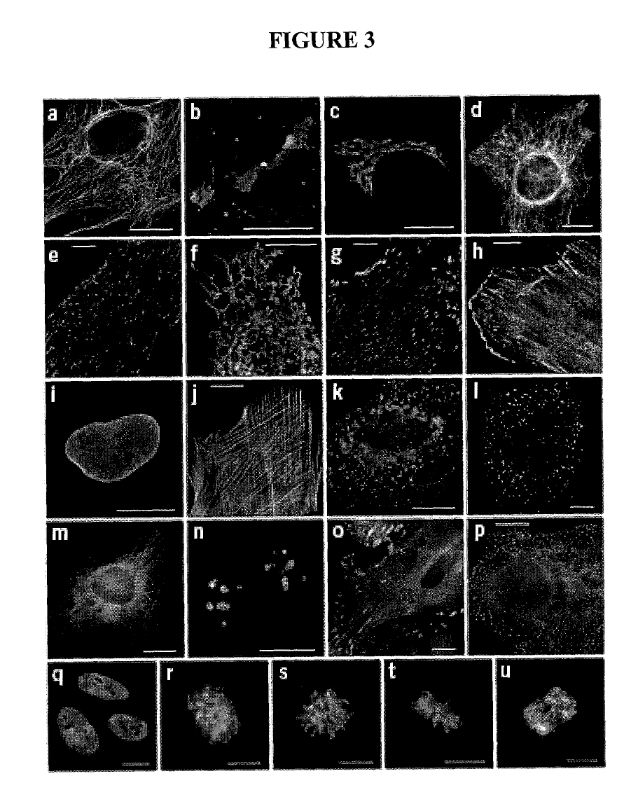

Use of mOrange2 for the construction of fusion proteins and use in localization studies. To confirm the fusion tolerance and targeting functionality of mOrange2 in a wide range of host protein chimeras, a series of 20 mOrange2 fusion constructs to both the C- and N-Terminus of the FP were constructed. In all cases, the localization patterns of the fusion proteins were similar to those previously or concurrently confirmed with AvGFP fusions (mEGFP and mEmerald) (see FIG. 3). Fusions of mOrange2 to histone H2B were observed not to hinder successful cell division as all phases of mitosis were present in cultures expressing this construct (FIG. 3q-u). mOrange2 also performed well as a fusion to the microtubule (+) end binding protein, EB3 (FIG. 3e) where it could be observed tracing the path of growing microtubules in time-lapse image sequences. Thus, mOrange2 is expected to perform as well as highly validated fluorescent proteins such as mEGFP in fusion constructs and for the use in localization studies.

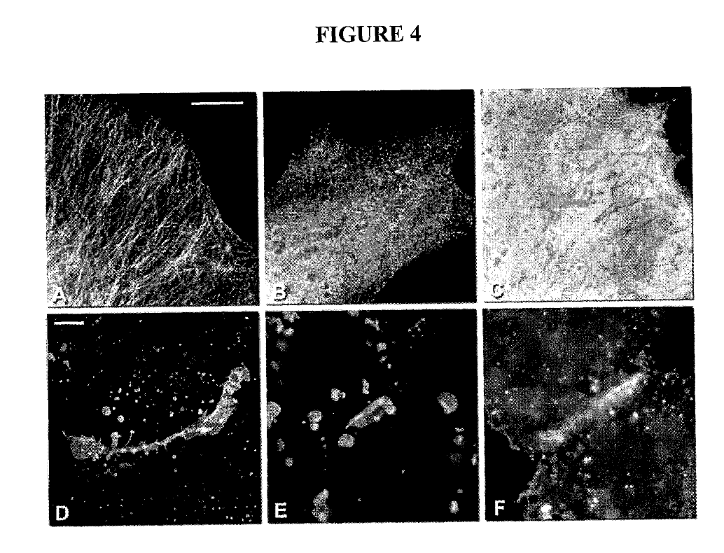

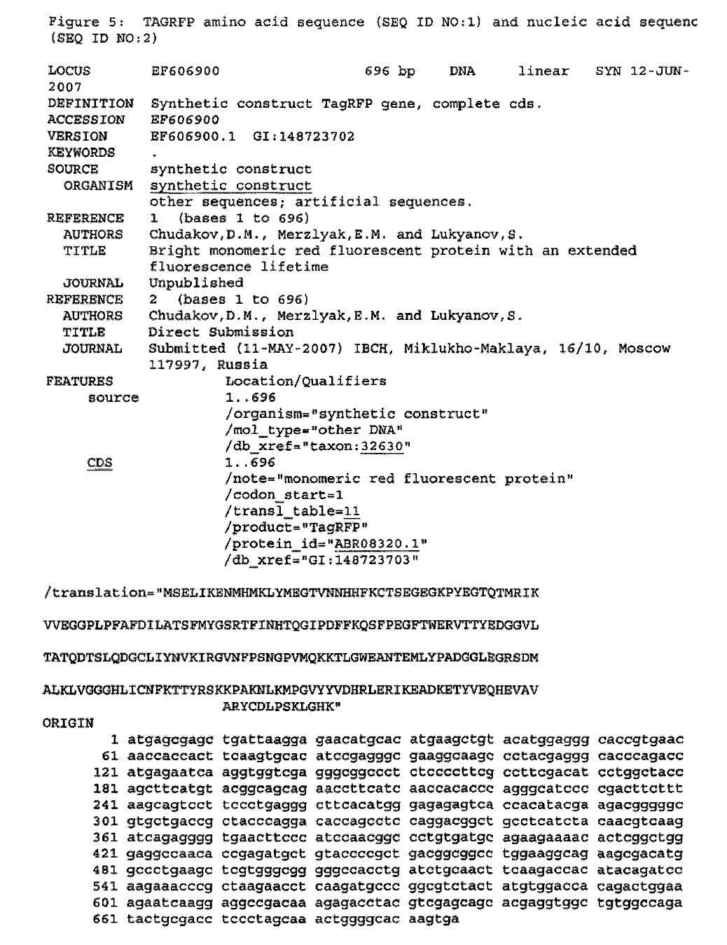

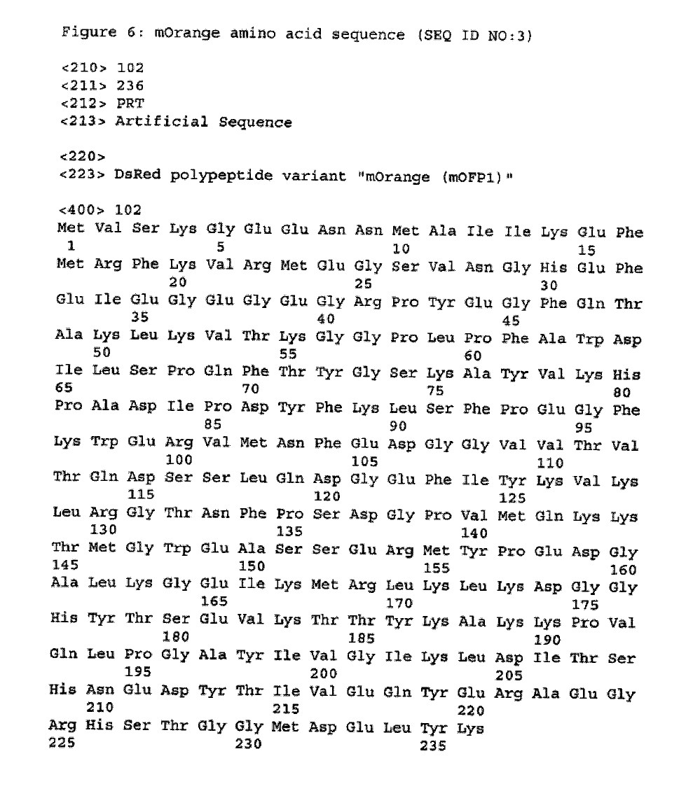

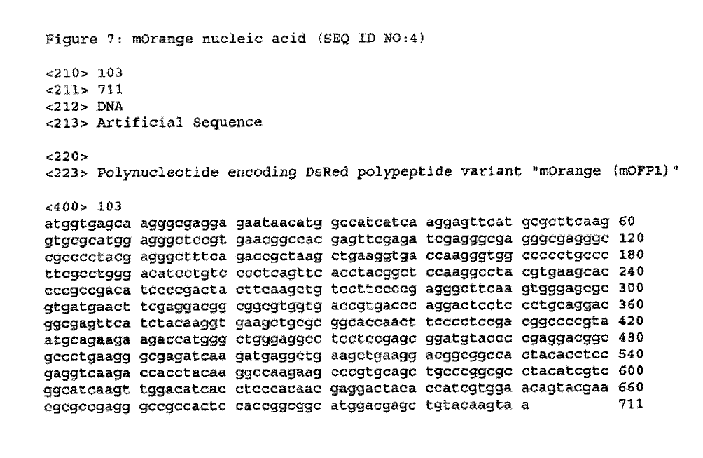

In order to compare the targeting capabilities of mOrange2 to other FPs in the orange spectral class, fusions of mKusabira Orange (mKO) and tdTomato to human α-Tubulin and rat α-1 connexin-43 were constructed and imaged in HeLa cells along with identical fusions to mOrange2 (FIG. 4). Because they are tightly packed in ordered tubulin filaments, FP fusions to α-Tubulin often do not localize properly if any degree of oligomeric character is present in the FP or if the construct experiences steric hindrance due to the size and/or folding behavior of the FP. Similarly, connexin-43 fusions are also sensitive to FP structural parameters in localization experiments.

Fusions of mOrange2 to α-Tubulin localize as expected to produce discernable microtubule filaments (FIG. 4a), but the same construct substituting mKO for mOrange2 exhibits punctate behavior that obscures the identification of any tendency to form filaments (FIG. 4b). The tdTomato-α-Tubulin fusion shows no evidence of localization and produces patterns reminiscent of whole-cell expression by the FP without a fusion partner (note the dark outlines of mitochondria in the cytoplasm: FIG. 4c). Fusions of mOrange2 with rat α-1 connexin-43 are assembled in the endoplasmic reticulum and traffic through the Golgi complex before being translocated to the plasma membrane and properly assembled into functional gap junctions (FIG. 4d). In contrast, mKO fusions with connexin-43 produce extraordinarily large cytoplasmic vesicles and form less clearly defined and much smaller gap junctions (FIG. 4e). tdTomato-connexin-43 fusions form aggregates in the cytoplasm accompanied by widespread labeling of the membrane with no apparent trafficking patterns through the endoplasmic reticulum and Golgi complex. In addition, the fusion does not form morphologically distinct gap junctions, but occasionally will produce regions of brighter fluorescence where plasma membranes of neighboring cells overlap (FIG. 4f).

### Example 5

Selection of a photostable TagRFP variant. While the recently developed orange-red monomer TagRFP is a promising choice as a FRET acceptor and for multicolor imaging, we have found that, contrary to the original report, its photostability is still far from optimal. In both standard arc lamp photobleaching and newer laser scanning confocal assays, it was determined that TagRFP bleaches approximately 3-fold faster than mCherry (see FIG. 1d, Table 1, and FIG. 9). Thus, this protein was chosen as another starting point to validate the novel photostability selection assay of the invention. Rather than using random mutagenesis, rational design of a mutant library was first implemented, guided by the crystal structure of the closely-related protein eqFP611 (Petersen, J. et al., J Biol Chem, 278:44626-44631 (2003)) (FIG. 15). With the rationale that chromophore-interacting residues could influence photostability, saturation mutagenesis was performed on S158 and L199, two residues proximal to the TagRFP chromophore. This library was then screened in bacteria with the solar simulator-based assay, this time taking images of the plates before and after bleaching to select those colonies that displayed high absolute brightness and a high ratio of post-bleach to pre-bleach fluorescence emission.

From the created directed library, one clone was identified, TagRFP S158T (designated “TagRFP-T”), which has a photobleaching half-time of 337 seconds by standard assay, making it approximately 9-fold more photostable than TagRFP (see FIG. 1d for arc lamp bleaching curves, FIG. 9 for laser scanning confocal bleaching curves, and Table 1 for detailed properties). TagRFP-T possesses identical excitation and emission wavelength, quantum yield, and maturation time to TagRFP, with only a slightly lower extinction coefficient (81,000 versus 98,000 M⁻¹·cm⁻¹) and a higher fluorescence pKa (4.6 versus 3.1). The benefit of increased photostability should offset the small decrease in brightness and increase in acid sensitivity in most practical contexts. As with mOrange2, it was verified that TagRFP-T remains monomeric by gel filtration. Since the S158T mutation is internal, it is likely that TagRFP-T will perform nearly identically as TagRFP when used as a fusion tag.

Photobleaching of TagRFP and TagRFP-T under oxygen-free conditions revealed that, unlike mOrange2, TagRFP-T's photobleaching remains oxygen-sensitive (see FIG. 2d and Table 1). However, like mOrange and mOrange2, the oxygen-free bleaching half-time for TagRFP is similar to the ambient oxygen bleaching half-time for TagRFP-T. TagRFP and TagRFP-T were compared as fusions to H2B expressed in living cells under confocal illumination (see FIG. 9 and Table 1). Consistent with previous results, TagRFP-T had a photobleaching half-time approximately 9-fold greater than that of TagRFP.

### Example 6

Plasmid construction. Synthetic DNA oligonucleotides for cloning and library construction were purchased from Integrated DNA Technologies (Coralville, Iowa). PCR products and products of restriction digests were purified by gel electrophoresis and extraction using the QIAquick™ gel extraction kit (QIAGEN, Valencia, Calif.). Plasmid DNA was purified from overnight cultures by using QIAprep Spin Miniprep kit (QIAGEN, Valencia, Calif.). Restriction endonucleases were purchased from either Invitrogen or New England Biolabs. DNA was purified from the remaining pellets by QIAprep spin column (Qiagen) and submitted for sequencing.

For mammalian expression, All mOrange2 expression vectors were constructed using C1 and N1 (Clontech-style) cloning vectors. The FP was amplified with a 5′ primer encoding an Age1 site and a 3′ primer encoding either a BspEI (C1) or Not1 (N1) site. The purified and digested PCR products were ligated into similarly digested EGFP-C1 and EGFP-NI cloning vector backbones. To generate fusion vectors, the appropriate cloning vector and an EGFP fusion vector were digested, either sequentially or doubly, with the appropriate enzymes and ligated together after gel purification. Thus, to prepare mOrange2 N-Terminal fusions, the following digests were performed: human non-muscle α-actinin, EcoRI and NotI (vector source, Tom Keller, FSU); human cytochrome C oxidase subunit VIII, BamHI and NotI (mitochondria, Clontech); rat α-1 connexin-43 and β-2 connexin-26, EcoRI and BamHI (Matthias Falk, Lehigh University); human histone H2B, BamHI and NotI (George Patterson, NIH); N-Terminal 81 amino acids of human β-1,4-galactosyltransferase, BamHI and NotI (Golgi, Clontech); human microtubule-associated protein EB3, BamHI and NotI (Lynne Cassimeris, Lehigh University); human vimentin, BamHI and NotI (Robert Goldman, Northwestern University); human keratin 18, EcoRI and NotI (Open Biosystems, Huntsville, Ala.); chicken paxillin, EcoRI and NotI (Alan Horwitz, University of Virginia); rat lysosomal membrane glycoprotein 1, AgeI and NheI (George Patterson, NIH). To prepare mOrange2 C-Terminal fusions, the following digests were performed: human β-actin, NheI and BglII (Clontech); human α-Tubulin, NheI and BglII (Clontech); human light chain clathrin, NheI and BglII (George Patterson, NIH); human lamin B1, NheI and BglII (George Patterson, NIH); human fibrillarin, AgeI and BglII (Evrogen); human vinculin, NheI and EcoRI (Open Biosystems, Huntsville, Ala.); peroximal targeting signal 1 (PTS1-peroxisomes), AgeI and BspEI (Clontech); human RhoB GTPase with an N-Terminal c-Myc epitope tag (endosomes), AgeI and BspEI (Clontech). DNA for mammalian transfection was prepared using the Plasmid Maxi kit (QIAGEN).

### Example 7

Mutagenesis and screening. mOrange² was used as the initial template for library construction by random mutagenesis. Error-prone PCR was performed using the GeneMorph II kit (Stratagene) following the manufacturer's protocol, using primers containing BamHI and EcoRI sites as previously described (Shaner, N. C. et al., Nat Biotechnol, 22:1567-1572 (2004)). Error-prone PCR products were digested with BamHI and EcoRI and ligated into a modified pBAD vector (Invitrogen) or a constitutive bacterial expression vector pNCS, both of which encode an N-Terminal 6×His tag and linker. Site-directed mutagenesis was performed using the QuikChange II kit (Stratagene) following the manufacturer's protocol. Chemically competent or electrocompetent Escherichia coli strain LMG194 (Invitrogen) were transformed with libraries and grown overnight on LB/agar supplemented with 50 μg/mL ampicillin (Sigma) and 0.02% (wt/vol) L-arabinose (Fluka) (for pBAD-based libraries) at 37° C. Whole plates of bacteria were photobleached for 10 to 120 minutes on a solar simulator with 1600 W mercury arc lamp (Spectra-Physics) using a home-built water-cooled aluminum block to prevent heating. Infrared and ultraviolet wavelengths were removed by a dichroic mirror and remaining visual spectrum light was filtered through 10 cm square bandpass filters (Chroma) appropriate to the fluorescent protein being bleached (540/30 nm for mOrange- and TagRFP-based libraries or 568/40 nm for mApple libraries). Final light intensities produced by the solar simulator were measured by a miniature integrating-sphere detector (SPD024 head and ILC 1700 meter, International Light Corp.) to be 95 mW/cm² for the 540/30 filter and 141 mW/cm² for the 568/40 filter. Plates were examined by eye or imaged using a UVP imaging system using 535/45 nm excitation and 605/70 nm emission filters. Colonies maintaining bright fluorescence after photobleaching and/or those with high post- to pre-bleach fluorescence ratios were cultured for 8 h in 2 ml Luria-Bertani (LB) medium supplemented with 100 μg/mL ampicillin, and then culture volume was increased to 4 ml with additional LB supplemented with ampicillin and 0.2% (wt/vol) L-arabinose to induce fluorescent protein expression and were grown overnight. A fraction of each cell pellet was extracted with P-BER II (Pierce) and spectra were obtained using a Safire 96-well plate reader with monochromators (TECAN). When screening for photostable variants, spectra were obtained before and after photobleaching extracted protein on the solar simulator.

### Example 8

Protein production and characterization. Fluorescent proteins were expressed from pBAD vectors in E. coli strain LMG194, purified, and characterized as described (Shaner, N. C. et al., Nat Biotechnol, 22:1567-1572 (2004)). Photobleaching measurements were performed on aqueous droplets of purified protein under oil as described (Shaner, N. C. et al., Nat Biotechnol, 22:1567-1572 (2004); Shaner, N. C. et al., Nat Methods, 2:905-909 (2005)). To determine if the presence of molecular oxygen influenced bleaching, we performed our standard bleaching experiment before and after equilibrating the entire bleaching apparatus under humidified N₂.

### Example 9

Mass spectrometry analysis. Parallel samples of purified mOrange were prepared without bleaching and with 60 minutes bleaching on the solar simulator, and dialyzed into 200 mM ammonium bicarbonate pH 8.5. Samples were then digested with LysC (Wako Biochemicals) which cuts at the C-Terminal side of lysine, or AspN (Roche Diagnostics) which cuts at the N-Terminal side of aspartic acid. For the LysC digests, protein was denatured in 6 M guanidinium HCl with incubation in a 72° C. water bath for 2 minutes, followed by addition of LysC enzyme at a 30:1 protein to enzyme ratio, and incubation for 18 hours at 36° C. For the AspN digests, protein was denatured in 8 M urea with incubation in a 90° C. water bath for 2 minutes, followed by addition of AspN enzyme at a 50:1 protein to enzyme ratio, an incubation for 18 hours at 36° C. Digested peptides were desalted with a C18 ZipTip (Millipore) to prepare the sample for matrix-assisted laser desorption/ionization (MALDI) mass spectrometry. The MALDI matrix used was α-cyanohydroxycinnamic acid (Fluka). Mass spectra were collected on an Voyager-DE STR MALDI-TOF (Applied Biosystems) using default tuning parameters.

### Example 10

Live Cell Imaging. HeLa epithelial (CCL-2, ATCC) and Grey fox lung fibroblast (CCL-168, ATCC) cells were grown in a 50:50 mixture of DMEM and Ham's F12 with 12.5% Cosmic calf serum (HyClone, Logan, Utah) and transfected with Effectene (QIAGEN). Imaging was performed in Delta-T culture chambers (Bioptechs, Butler, Pa.) under a humidified atmosphere of 5% CO₂ in air. All filters for fluorescence screening and imaging were purchased from Chroma Technology (Rockingham, Vt.), Omega Filters (Brattleboro, Vt.), and Semrock (Rochester, N.Y.). Fluorescence images in widefield mode were gathered using a Nikon (Melville, N.Y.) TE-2000 inverted microscope equipped with Omega QuantaMax™ filters and a Photometrics (Tucson, Ariz.) Cascade II camera or an Olympus (Lehigh Valley, Pa.) IX71 equipped with Semrock BrightLine™ filters and a Hamamatsu (Bridgewater, N.J.) ImagEM™ camera. Laser scanning confocal microscopy was performed on a Nikon C1Si and an Olympus FV1000, both equipped with helium-neon and diode lasers and proprietary filter sets to match fluorophore emission spectral profiles. Spinning disk confocal microscopy was performed on an Olympus DSU-IX81 equipped with a Lumen 200 illuminator (Prior, Boston, Mass.), Semrock filters, 10-position filter wheels driven by a Lambda 10-3 controller (Sutter, Novato, Calif.), and a Hamamatsu 9100-12 EMCCD camera. Cell cultures expressing FP fusions were fixed after imaging in 2% paraformaldehyde (EMS, Hatfield, Pa.) and washed several times in PBS containing 0.05 M glycine before mounting with a polyvinyl alcohol-based medium. Morphological features in all fusion constructs were confirmed by imaging fixed cell preparations on coverslips using a Nikon 80i upright microscope and ET-DsRed filter set (#4900; Chroma) coupled to a Hamamatsu Orca ER or a Photometrics CoolSNAP™ camera.HQ² camera.

### Example 11

Laser scanning confocal microscopy (LSCM) Live Cell Photobleaching. Laser scanning confocal microscopy photobleaching experiments were conducted with N-Terminal fusions of the appropriate FP to human histone H2B (6-residue linker) to confine fluorescence to the nucleus in order to closely approximate the dimensions of aqueous droplets of purified FPs used in widefield measurements. HeLa-S3 cells (average nucleus diameter=17 μm) were transfected with the H2B construct using Effectene (QIAGEN) and maintained in 5% CO₂ Bioptechs Delta-T imaging chambers for at least 36 hours prior to imaging. The chambers were transferred to a Bioptechs stage adapter, imaged at low magnification to ensure cell viability, and then photobleached using a 40× oil immersion objective (Olympus UPlan Apo, NA=1.00). Laser lines (543 nm, He—Ne and 488 nm, argon-ion) were adjusted to an output power of 50 μW, measured with a FieldMaxII-TO (Coherent) power meter equipped with a high-sensitivity silicon/germanium optical sensor (Coherent OP-2Vis). The instrument (Olympus FV300) was set to a zoom of 4×, a region of interest of 341.2 μm² (108×108 pixels), a photomultiplier voltage of 650 V, and an offset of 9% with a scan time of 0.181 seconds per frame. Nuclei having approximately the same dimensions and intensity under the fixed instrument settings were chosen for photobleaching assays. Fluorescence using the 543 laser was recorded with a 570 nm dichromatic mirror and 656 nn longpass barrier filter, whereas emission using the 488 laser was directly reflected by a mirror through a 510 nm longpass banier filter. The photobleaching half-times for LSCM imaging were calculated as the time required to reduce the scan-averaged emission rate to 50% from an initial emission rate of 1000 photons/s per fluorescent protein chromophore. Briefly, the average photon flux (photons/(s·m²)) over the scanned area of interest was calculated thus:

$$Φ = \frac{P}{EA} = \frac{P λ}{hcA}$$

where P is the output power of the laser measured at the objective in Joules/sec, A is the scanned area in m², and E=hc/λ is the energy of a photon in Joules at the laser wavelength (either 543 nm or 488 nm). The optical cross section (in cm²) of a fluorescent protein chromophore is given by:

$$σ ( λ ) = \frac{( 1000 {cm}^{3} ) ( ln 10 ) ∈ ( λ )}{6.023 x 10 E 23 / mole}$$

where ε(λ) is the extinction coefficient of the fluorescent protein at the laser wavelength in M⁻¹·cm⁻¹. Thus, the scan-average excitation rate per fluorescent molecule is given by:

X=Φσ(λ).

Thus, the time to bleach from an initial scan-averaged rate of 1000 photons/s to 500 photons/s is:

t_{1/2}=(t_{raw}XQ)/(1000 photons/s)

where t_{raw} is the measured photobleaching half-Time and Q is the fluorescent protein quantum yield. To get full bleaching curves, we simply scale the raw time coordinates by the factor XQ/(1000 photons/s) and normalize the intensity coordinate to 1000 photons/s initial emission rate.

### Example 12

Reversible photoswitching assays. Observation that newly engineered photostable fluorescent protein variants exhibited varying degrees of reversible photoswitching led to the exploration of this phenomenon in other commonly used fluorescent proteins. To qualitatively measure this behavior, histone H2B fusions to each fluorescent protein were expressed and imaged in HeLa-S3 cells by widefield and laser scanning confocal microscopy (LSCM). For both widefield and LSCM imaging, cells were exposed to constant illumination without neutral density filters (widefield) or with 25-100% laser power (LSCM) (corresponding to excitation intensities between 32 and 151 W/cm² for widefield and between 49 and 637 W/cm² (scan-averaged) for LSCM) until they had dimmed to between 75% and 50% initial fluorescence intensity. The cells were then allowed to recover in darkness for 1 to 2 minutes, after which time they were re-imaged. Any recovery of fluorescence could not be due to diffusion from non-illuminated regions, because the histone H2B fusions were confined within nuclei that were entirely within the bleached area. The percent recovery (% REC) of the peak initial fluoresence was calculated as:

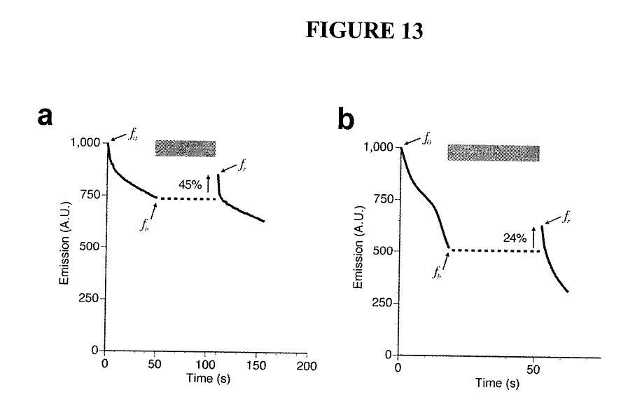

% REC=(fᵣ−f_{bl})/(fₒ−f_{bl})

where fₒ is the peak initial fluorescence, f_{bl} is the post-bleach fluorescence, and fᵣ is the post-dark recovery fluorescence. See FIG. 13 for an example of the behavior of EGFP under widefield and confocal illumination. Results for a wide variety of FPs are reported in Table 3 below. While these data strongly suggest that reversible photoswitching is a common feature among fluorescent proteins, these data are not intended to be quantitative; further in-depth investigation of this phenomenon under a wider variety of experimental conditions will be necessary to fully characterize this effect and its possible implications in any given experiment.

TABLE 3
Summary of reversible photoswitching data.

| | | |
|---|---|---|
| | % recovery, widefield | % recovery, confocal |
| Protein^{a} | (excitation intensity)^{b} | (excitation intensity)^{b} |

| TagRFP-T | 13 | (96 W/cm₂) | 30 | (181 W/cm₂) |
|---|---|---|---|---|
| TagRFP | 4 | (108 W/cm₂) | 14 | (181 W/cm₂) |
| mOrange2 | 6 | (96 W/cm₂) | 4.1 | (181 W/cm₂) |
| mCherry | 14 | (151 W/cm₂) | 4 | (181 W/cm₂) |
| tdTomato | ND^{c} | | 0 | (181 W/cm₂) |
| mKO | 4 | (96 W/cm₂) | 18 | (181 W/cm₂) |
| mKate | 0 | (155 W/cm₂) | 6.6 | (181 W/cm₂) |
| mCerulean | 113 | (50 W/cm₂) | 10 | (230 W/cm₂) |
| mVenus | 23 | (32 W/cm₂) | 47 | (225 W/cm₂) |
| EYFP | 9.8 | (32 W/cm₂) | 31 | (225 W/cm₂) |
| Citrine | 5.9 | (32 W/cm₂) | 38 | (441 W/cm₂) |
| YPet | 10 | (32 W/cm₂) | 24 | (49 W/cm₂) |
| Topaz | 16 | (32 W/cm₂) | 65 | (225 W/cm₂) |
| mEGFP | 45 | (54 W/cm₂) | 24 | (637 W/cm₂) |

^{a}Fluorescent proteins fused to histone H2B and expressed in HeLa-S3 cells (see text above).
^{b}Percent dark recovery of fluorescence after dimming to between 50 and 75% initial peak fluorescence, followed by 1 to 2 minutes darkness; see text above for complete description and Figure D above for representative mEGFP traces. Excitation intensity, as measured at the objective, is shown in parentheses (scan-averaged for LSCM).
^{c}ND = not determined

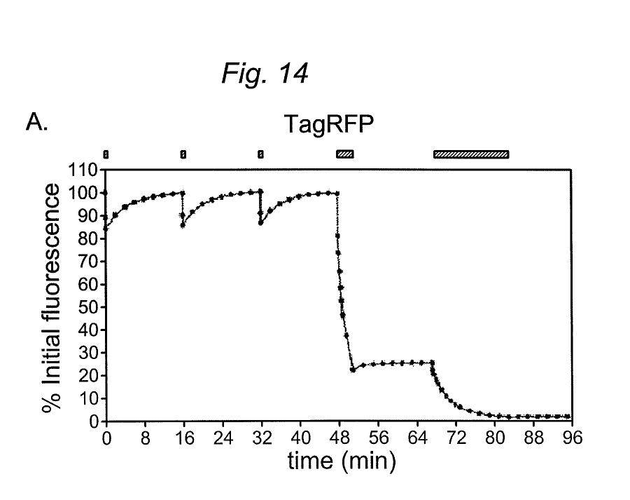

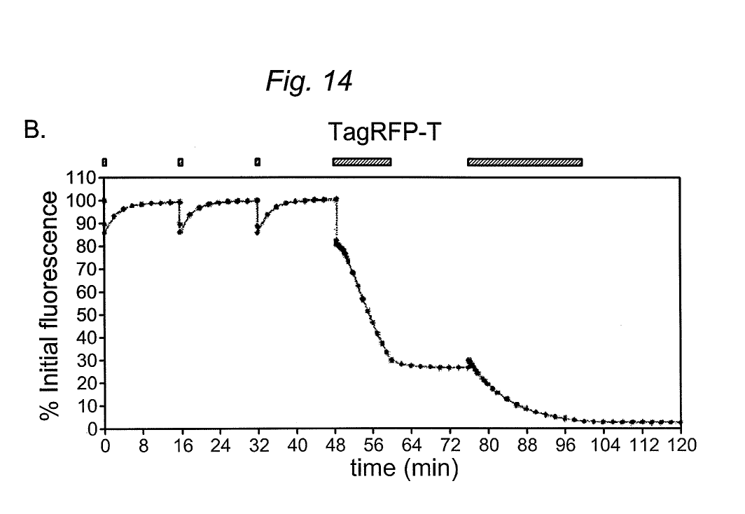

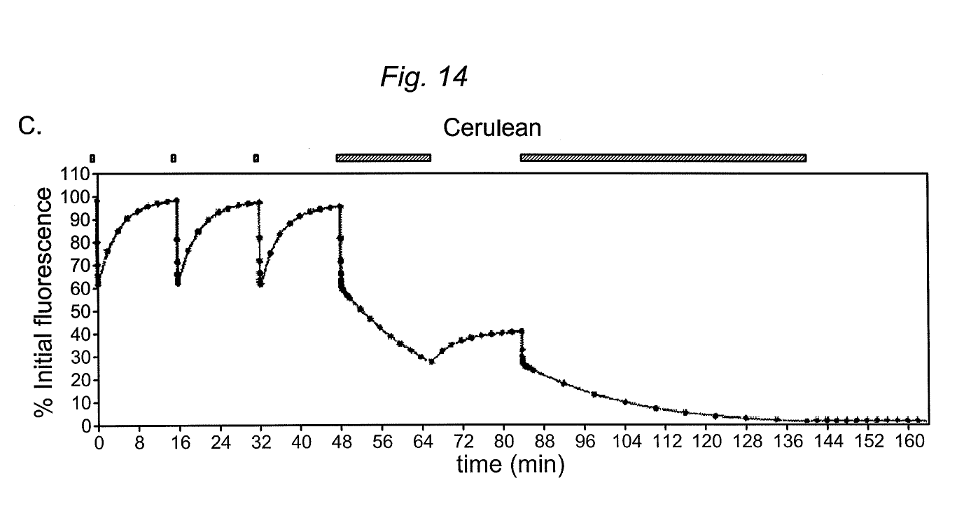

To more precisely characterize the degree of reversible photoswitching in three representative proteins (TagRFP, TagRFP-T, and Cerulean), aqueous droplets of purified protein under oil were bleached on a microscope at ambient temperatures with xenon arc lamp illumination through a 540/25 filter (for TagRFP and TagRFP-T) or 420/20 nm filter (for Cerulean) without neutral density filters for short (˜2 to 10 s) or long (˜2 to 10 min) intervals, and allowed to recover in the dark while fluorescence intensity was measured with 50 ms exposures (FIG. 14). All three proteins were able to recover to nearly 100% after very short periods of bleaching, and to a lesser degree after longer periods. Once again, these data strongly indicate the need for further investigation of this phenomenon in all commonly used fluorescent proteins.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A photostable fluorescent protein variant comprising an amino acid sequence that is at least 85% identical to SEQ ID NO:12, comprising an F99Y mutation, wherein said variant is more photostable than a polypeptide of SEQ ID NO:12.

2. The photostable fluorescent protein variant of claim 1, wherein said variant further comprises a Q64H mutation.

3. The photostable fluorescent protein variant of claim 1, wherein said variant further comprises at least one of a Lys at residue 160 and an Asp at residue 196.

4. A fusion protein comprising a photostable fluorescent protein of claim 1 operatively linked to a protein of interest.

5. A photostable tandem fluorescent protein comprising a first photostable fluorescent protein of claim 1 operatively linked to a second fluorescent protein.

6. The photostable tandem fluorescent protein of claim 5, wherein said first photostable fluorescent protein and said second fluorescent protein are capable of performing intramolecular FRET.

7. A nucleic acid encoding a photostable fluorescent protein of claim 1.

8. A vector comprising the nucleic acid of claim 7.

9. A host cell comprising the vector of claim 8.

10. A method of detecting a protein of interest, the method comprising the steps of:
(a) expressing a fusion protein of said protein of interest and a photostable fluorescent protein variant comprising an amino acid sequence that is at least 85% identical to SEQ ID NO:12, comprising an F99Y mutation, wherein said variant is more photostable than a polypeptide of SEQ ID NO:12; and
(b) detecting the fluorescence of said fusion protein, thereby detecting a protein of interest.

11. A method of detecting the cellular localization of a protein of interest, the method comprising the steps of:
(a) expressing in a cell a fusion protein of said protein of interest and a photostable fluorescent protein variant comprising an amino acid sequence that is at least 85% identical to SEQ ID NO:12, comprising an F99Y mutation, wherein said variant is more photostable than a polypeptide of SEQ ID NO:12;
(b) detecting the fluorescence of said fusion protein; and
(c) determining the cellular location of said fluorescence, thereby detecting the cellular localization of a protein of interest.

12. A method of detecting the motility of a protein of interest, the method comprising the steps of:
(a) expressing in a cell a fusion protein of said protein of interest and a photostable fluorescent protein variant comprising an amino acid sequence that is at least 85% identical to SEQ ID NO:12, comprising an F99Y mutation, wherein said variant is more photostable than a polypeptide of SEQ ID NO:12;
(b) performing time-sequential observations of fluorescence in said cell; and
(c) detecting differences in said fluorescence between said time-sequential observations, thereby detecting protein motility of a protein of interest.

13. A method of detecting an interaction between a first protein of interest and a second protein of interest, the method comprising the steps of:
(a) contacting a first fusion protein of said first protein of interest and a photostable fluorescent protein variant comprising an amino acid sequence that is at least 85% identical to SEQ ID NO:12, comprising an F99Y mutation, wherein said variant is more photostable than a polypeptide of SEQ ID NO:12, with a second fusion protein of said second protein of interest and a fluorescent protein, wherein said first fluorescent protein and said second fluorescent protein are capable of performing intermolecular FRET; and
(b) detecting a change in fluorescence, thereby detecting an interaction between said first protein of interest and said second protein of interest.

14. A photostable fluorescent protein variant comprising an amino acid sequence that is at least 85% identical to SEQ ID NO:3, comprising mutations selected from the group consisting of F99Y, Q64H/F99Y, Q64H/F99Y/E160K, Q64H/F99Y/G196D, and Q64H/F99Y/E160K/G196D, wherein said variant is more photostable than a polypeptide of SEQ ID NO:3.

15. The photostable fluorescent protein variant of claim 14, wherein said variant comprises an amino acid sequence of SEQ ID NO:11.

16. A nucleic acid encoding a photostable fluorescent protein of claim 15.

17. A photostable fluorescent protein variant comprising an amino acid sequence that is at least 85% identical to SEQ ID NO:18, comprising an S158T mutation, wherein said variant is more photostable than a polypeptide of SEQ ID NO:18.

18. A photostable fluorescent protein variant comprising an amino acid sequence that is at least 85% identical to SEQ ID NO:1, comprising an S158T mutation, wherein said variant is more photostable than a polypeptide of SEQ ID NO:1.

19. The photostable fluorescent protein of claim 18, wherein said photostable protein comprises an amino acid sequence of SEQ ID NO:7.

20. A fusion protein comprising the photostable fluorescent protein of claim 17.

21. A photostable tandem fluorescent protein comprising a first photostable fluorescent protein of claim 17 operatively linked to a second fluorescent protein.

22. The photostable tandem fluorescent protein of claim 21, wherein said first photostable fluorescent protein and said second fluorescent protein are capable of performing intramolecular FRET.

23. A nucleic acid encoding a photostable fluorescent protein of claim 17.

24. A fluorescent protein variant comprising an amino acid sequence that is at least 85% identical to SEQ ID NO:3, comprising mutations selected from:
(a) G40A/T66M/A71V/A73V/V104I/V105I/T106H/T108N/E117V/G159S/M163K/T174A/G196D; and
(b) R17H/G40A/T66M/A71V/A73I/K92R/V104I/V105I/T106H/T108N/E117V/S147E/G159S/M163K/T174A/S175A/G196D/T202V.

25. The fluorescent protein variant of claim 24, wherein said variant comprises the amino acid sequence of SEQ ID NO:9.

26. The fluorescent protein variant of claim 24, wherein said variant comprises the amino acid sequence of SEQ ID NO:10.

27. A fusion protein comprising the photostable fluorescent protein of claim 24.

28. A photostable tandem fluorescent protein comprising a first photostable fluorescent protein of claim 24 operatively linked to a second fluorescent protein.

29. The photostable tandem fluorescent protein of claim 28, wherein said first photostable fluorescent protein and said second fluorescent protein are capable of performing intramolecular FRET.

30. A method of evolving a photostable fluorescent protein variant, the method comprising the steps of:
(a) mutating a nucleic acid encoding a fluorescent protein; and
(b) performing a selection assay for a mutated fluorescent protein with increased photostability as compared to the parent fluorescent protein,
wherein steps (a) and (b) may optionally be repeated one or more times, thereby generating a photostable fluorescent protein variant.

31. The method of claim 30, wherein said selection assay comprises the steps of:
(a) photobleaching a plurality of colonies expressing mutants of the fluorescent protein being evolved for a predetermined set of time; and
(b) selecting the brightest post-bleach colonies, thereby generating a photostable fluorescent protein variant.

32. A photostable fluorescent protein variant generated by a method of claim 30.


SEQUENCE LISTING (23 sequences)

SEQ ID NO: 1 - protein, 231 aa, Artificial Sequence
  TagRFP, monomeric red fluorescent protein derived from eqFP578
MSELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRSDMALKLVGGGHLICNFKTTYRSKKPAKNLKMPGVYYVDHRLERIKEADKETYVEQHEVAVARYCDLPSKLGHK

SEQ ID NO: 2 - DNA, 696 bp, Artificial Sequence
  TagRFP, monomeric red fluorescent protein coding sequence derived from eqFP578
atgagcgagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaagtga

SEQ ID NO: 3 - protein, 236 aa, Artificial Sequence
  mOrange, first-generation monomeric orange fluorescent protein (mRFP1) “mFruit” variant
MVSKGEENNMAIIKEFMRFKVRMEGSVNGHEFEIEGEGEGRPYEGFQTAKLKVTKGGPLPFAWDILSPQFTYGSKAYVKHPADIPDYFKLSFPEGFKWERVMNFEDGGVVTVTQDSSLQDGEFIYKVKLRGTNFPSDGPVMQKKTMGWEASSERMYPEDGALKGEIKMRLKLKDGGHYTSEVKTTYKAKKPVQLPGAYIVGIKLDITSHNEDYTIVEQYERAEGRHSTGGMDELYK

SEQ ID NO: 4 - DNA, 711 bp, Artificial Sequence
  mOrange, first-generation monomeric orange fluorescent protein (mRFP1) “mFruit” variant coding sequence
atggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcagttcacctacggctccaaggcctacgtgaagcaccccgccgacatccccgactacttcaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacacctccgaggtcaagaccacctacaaggccaagaagcccgtgcagctgcccggcgcctacatcgtcggcatcaagttggacatcacctcccacaacgaggactacaccatcgtggaacagtacgaacgcgccgagggccgccactccaccggcggcatggacgagctgtacaagtaa

SEQ ID NO: 5 - protein, 225 aa, Discosoma sp.
  wild-type corallimorph anthozoan red fluorescent protein DsRed (wtDsRed, drFP583)
MRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 6 - protein, 236 aa, Artificial sequence
  mCherry, first-generation monomeric red fluorescent protein (mRFP1) “mFruit” variant
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 7 - protein, 244 aa, Artificial Sequence
  photostable TagRFP variant TagRFP-T (TagRFP S158T), monomeric red fluorescent protein derived from eqFP578
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTDMALKLVGGGHLICNFKTTYRSKKPAKNLKMPGVYYVDHRLERIKEADKETYVEQHEVAVARYCDLPSKLGHKLNGMDELYK

SEQ ID NO: 8 - protein, 236 aa, Artificial Sequence
  photostable variant mApple0.1, monomeric red fluorescent protein derived from mOrange
MVSKGEENNMAIIKEFMRFKVRMEGSVNGHEFEIEGEGEGRPYEAFQTAKLKVTKGGPLPFAWDILSPQFQYGSKAYVKHPADIPDYFKLSFPEGFKWERVMNFEDGGVVTVTQDSSLQDGEFIYKVKLRGTNFPSDGPVMQKKTMGWEASSERMYPEDGALKGEIKMRLKLKDGGHYTSEVKTTYKAKKPVQLPGAYIVGIKLDITSHNEDYTIVEQYERAEGRHSTGGMDELYK

SEQ ID NO: 9 - protein, 236 aa, Artificial Sequence
  photostable variant mApple0.5, monomeric red fluorescent protein derived from mOrange
MVSKGEENNMAIIKEFMRFKVRMEGSVNGHEFEIEGEGEGRPYEAFQTAKLKVTKGGPLPFAWDILSPQFMYGSKVYIKHPADIPDYFKLSFPEGFKWERVMNFEDGGIIHVNQDSSLQDGVFIYKVKLRGTNFPSDGPVMQKKTMGWEASSERMYPEDGALKSEIKKRLKLKDGGHYASEVKTTYKAKKPVQLPGAYIVDIKLDITSHNEDYTIVEQYERAEGRHSTGGMDELYK

SEQ ID NO: 10 - protein, 236 aa, Artificial Sequence
  photostable variant mApple, monomeric red fluorescent protein derived from mOrange
MVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEASEERMYPEDGALKSEIKKRLKLKDGGHYAAEVKTTYKAKKPVQLPGAYIVDIKLDIVSHNEDYTIVEQYERAEGRHSTGGMDELYK

SEQ ID NO: 11 - protein, 236 aa, Artificial Sequence
  photostable variant mOrange2, monomeric orange fluorescent protein derived from mOrange
MVSKGEENNMAIIKEFMRFKVRMEGSVNGHEFEIEGEGEGRPYEGFQTAKLKVTKGGPLPFAWDILSPHFTYGSKAYVKHPADIPDYFKLSFPEGFKWERVMNYEDGGVVTVTQDSSLQDGEFIYKVKLRGTNFPSDGPVMQKKTMGWEASSERMYPEDGALKGKIKMRLKLKDGGHYTSEVKTTYKAKKPVQLPGAYIVDIKLDITSHNEDYTIVEQYERAEGRHSTGGMDELYK

SEQ ID NO: 12 - protein, 225 aa, Artificial Sequence
  Discosoma corallimorph anthozoan first- generationmonomeric red fluorescent protein (DsRed) variant mRFP1
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 13 - protein, 231 aa, Artificial Sequence
  photostable TagRFP variant TagRFP-T0.1, monomeric red fluorescent protein derived from eqFP578
MSELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTDMALKLVGGGHLICNFKTTYRSKKPAKNLKMPGVYYVDHRLERIKEADKETYVEQHEVAVARYCDLPSKLGHK

SEQ ID NO: 14 - protein, 231 aa, Entacmaea quadricolor
  wild-type eqFP611 monomeric red fluorescent protein
MNSLIKENMRMMVVMEGSVNGYQFKCTGEGDGNPYMGTQTMRIKVVEGGPLPFAFDILATSFMYGSKTFIKHTKGIPDFFKQSFPEGFTWERVTRYEDGGVFTVMQDTSLEDGCLVYHAKVTGVNFPSNGAVMQKKTKGWEPNTEMLYPADGGLRGYSQMALNVDGGGYLSCSFETTYRSKKTVENFKMPGFHFVDHRLERLEESDKEMFVVQHEHAVAKFCDLPSKLGRL

SEQ ID NO: 15 - protein, 238 aa, Aequorea victoria
  wild-type Pacific Northwest cnidarian jellyfish green fluorescent protein (GFP, AvGFP)
MSKGEELFTGVVPILVELDGDVNGQKFSVRGEGEGDATYGKLTLKFICTTGKLPVPWPTLVTTFSYGVQCFSRYPDHMKQHDFLKSAMPEGYVQERTIFYKDDGNYKTRAEVKFEGDTLVNRIELKGIDFKEDGNILGHKMEYNYNSHNVYIMGDKPKNGIKVNFKIRHNIKDGSVQLADHYQQNTPIGDGPVLLPDNHYLSTQSALSQDPHGKRDHMVLLEFVTSAGITHGMDELYK

SEQ ID NO: 16 - protein, 5 aa, Artificial Sequence
  amino acid insertion after residue position 6 in some of the mFruits, including mOrange and mStrawberry
ENNMA

SEQ ID NO: 17 - protein, 5 aa, Artificial Sequence
  amino acid insertion after residue position 6 in some of the mFruits, including mCherry
EDNMA

SEQ ID NO: 18 - protein, 231 aa, Entacmaea quadricolor
  wild-type eqFP578 monomeric red fluorescent protein
MSELIKENMHMKLYMEGTVNNHHFKCTSEGERKPYEGTQTMKIKVVEGGPLPFAFDILATSFMYGSKTFINHTQGIPDLFKQSFPEGFTWERITTYEDGGVLTATQDTSLQNGCIIYNVKINGVNFPSNGSVMQKKTLGWEANTEMLYPADGGLRGHSQMALKLVGGGYLHCSFKTTYRSKKPAKNLKMPGFHFVDHRLERIKEADKETYVEQHEMAVAKYCDLPSKLGHR

SEQ ID NO: 19 - protein, 231 aa, Artificial Sequence
  photostable TagRFP variant TurboRFP, monomeric red fluorescent protein derived from eqFP578
MSELIKENMHMKLYMEGTVNNHHFKCTSEGEGKPYEGTQTMKIKVVEGGPLPFAFDILATSFMYGSKAFINHTQGIPDFFKQSFPEGFTWERITTYEDGGVLTATQDTSFQNGCIIYNVKINGVNFPSNGPVMQKKTRGWEANTEMLYPADGGLRGHSQMALKLVGGGYLHCSFKTTYRSKKPAKNLKMPGFHFVDHRLERIKEADKETYVEQHEMAVAKYCDLPSKLGHR

SEQ ID NO: 20 - protein, 12 aa, Artificial Sequence
  synthetic peptide linker for tandem fluorescent protein
GHGTGSTGSGSS

SEQ ID NO: 21 - protein, 13 aa, Artificial Sequence
  synthetic peptide linker for tandem fluorescent protein
RMGSTSGSTKGQL

SEQ ID NO: 22 - protein, 22 aa, Artificial Sequence
  synthetic peptide linker for tandem fluorescent protein
RMGSTSGSGKPGSGEGSTKGQL

SEQ ID NO: 23 - protein, 6 aa, Artificial Sequence
  synthetic peptide tag, polyhistidine tag
HHHHHH